SEQUENCE LISTING (63 sequences)

SEQ ID NO: 1 - protein, 372 aa, synthetic construct
  REGION 1..372: note = Lignin peroxidase
MAFKQLIVAISIALSLQVTQAVVLKDKRATCSNGASVGDESCCAWFDVLDDIQQNLFNGGQCGAEAHESIRLVFHDSIAISPAMEAQGKFGGGGADGSIILFDEIETAFHPNIGLDEVVNLQKPFIAKHGVTPGDFIAFAGAVAMSNCPGAPQMNFFTGRAPATQAAPDGLVPEPFHTVDQIIDRVNDAGQFDELELVWMLSAHSVAASNDVDPTVQGLPFDSTPGVFDSQFFVETQLRGVLFPGSGGNQGEVESGLAGEIRLQSDHTLARDSRTACEWQSFVNNQSKLTSDFQFIFLALTQLGQNPDAMTDCSAVIPISKPIPGNGPFSFFPAGKTSADVEQACASTPFPSLTTLPGPTTSVARIPPPPGA

SEQ ID NO: 2 - DNA, 1120 bp, synthetic construct
  misc_feature 1..1120: note = Lignin peroxidase
  source 1..1120: mol_type = other DNA
atggccttcaagcagctcatcgtcgcaatttccatcgcgctctctctccaggtcactcaagctgtcgtgctgaaggacaagcgcgccacctgctcaaacggtgccagcgtcggcgacgaatcgtgctgcgcatggttcgacgtcctcgacgacatccagcagaacctcttcaacggaggccaatgcggcgctgaagcccacgagtccatccgactcgtcttccacgactccatcgccatctctcctgcaatggaggcacaggggaagtttggtggtggaggtgccgacggctccatcattctcttcgatgagattgagaccgcattccacccgaacatcggtctcgacgaagtcgtcaatcttcagaagccattcatcgctaaacacggtgtcacccccggcgattttattgccttcgccggtgcagtcgccatgagcaactgccccggtgccccgcagatgaacttcttcactggtcgcgctcctgctacccaagccgcgcccgatggtctcgttcccgagccgttccacactgtcgaccagatcattgaccgtgtcaacgatgccggccagttcgatgagctcgagcttgtctggatgctctcggcccactctgtcgcggcctccaacgacgtcgacccgactgtccaaggcctgccgttcgactccactcccggcgtctttgactcccagttcttcgtcgagacccagctacgcggtgtgctcttccccggctctgggggcaaccaaggcgaggtcgagtctggactcgcgggtgaaatccgtcttcagtccgaccataccctcgcgcgcgactcgcgcacggcctgcgagtggcagtcctttgtcaacaaccagtcgaagctgacgagcgacttccagttcatcttcctcgcgctcacacagctcggccagaacccggacgcgatgaccgactgctcggccgtcattccgatctccaagcccatccccggcaacggcccgttctcgttcttccccgccggcaagaccagcgccgacgtcgagcaggcttgcgcgtccacccccttcccgagcctcacgactctccctggccccacgacttcggtcgctcgaatcccaccacctccaggtgcttaat

SEQ ID NO: 3 - protein, 153 aa, synthetic construct
  REGION 1..153: note = LMCO of Aspergillus flavus
MAPLKTLVALLSANVLTSVLAELVKFEVDLTWAKGSPDGNLRDMIFVNDQFPAPQLTLNQYDDVEFTVNNHMPFNATVHFHGIVQLNTPWSDGVPGLTQKPILPGGTFTYRWTATEYGTYWYHAHARSLMADGLYGAIWIKCVPRWPSILCKD

SEQ ID NO: 4 - DNA, 459 bp, synthetic construct
  misc_feature 1..459: note = LMCO of Aspergillus flavus
  source 1..459: mol_type = other DNA
atggcgccgctgaaaaccctggtggcgctgctgagcgcgaacgtgctgaccagcgtgctggcggaactggtgaaatttgaagtggatctgacctgggcgaaaggcagcccggatggcaacctgcgcgatatgatttttgtgaacgatcagtttccggcgccgcagctgaccctgaaccagtatgatgatgtggaatttaccgtgaacaaccatatgccgtttaacgcgaccgtgcattttcatggcattgtgcagctgaacaccccgtggagcgatggcgtgccgggcctgacccagaaaccgattctgccgggcggcacctttacctatcgctggaccgcgaccgaatatggcacctattggtatcatgcgcatgcgcgcagcctgatggcggatggcctgtatggcgcgatttggattaaatgcgtgccgcgctggccgagcattctgtgcaaagat

SEQ ID NO: 5 - protein, 422 aa, synthetic construct
  REGION 1..422: note = OleT
MATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 6 - DNA, 1269 bp, synthetic construct
  misc_feature 1..1269: note = OleT
  source 1..1269: mol_type = other DNA
atggcaacacttaagagggataagggcttagataatactttgaaagtattaaagcaaggttatctttacacaacaaatcagagaaatcgtctaaacacatcagttttccaaactaaagcactcggtggtaaaccattcgtagttgtgactggtaaggaaggcgctgaaatgttctacaacaatgatgttgttcaacgtgaaggcatgttaccaaaacgtatcgttaatacgctttttggtaaaggtgcaatccatacggtagatggtaaaaaacacgtagacagaaaagcattgttcatgagcttgatgactgaaggtaacttgaattatgtacgagaattaacgcgtacattatggcatgcgaacacacaacgtatggaaagtatggatgaggtaaatatttaccgtgaatctatcgtactacttacaaaagtaggaacacgttgggcaggcgttcaagcaccacctgaagatatcgaaagaatcgcaacagacatggacatcatgatcgattcatttagagcacttggtggtgcctttaaaggttacaaggcatcaaaagaagcacgtcgtcgtgttgaagattggttagaagaacaaattattgagactcgtaaagggaatattcatccaccagaaggtacagcactttacgaatttgcacattgggaagactacttaggtaacccaatggactcaagaacttgtgcgattgacttaatgaacacattccgcccattaatcgcaatcaacagattcgtttcattcggtttacacgcgatgaacgaaaacccaatcacacgtgaaaaaattaaatcagaacctgactatgcatataaattcgctcaagaagttcgtcgttactatccattcgttccattccttccaggtaaagcgaaagtagacatcgacttccaaggcgttacaattcctgcaggtgtaggtcttgcattagatgtttatggtacaacgcatgatgaatcactttgggacgatccaaatgaattccgcccagaaagattcgaaacttgggacggatcaccatttgaccttattccacaaggtggtggagattactggacaaatcaccgttgtgcaggtgaatggatcacagtaatcatcatggaagaaacaatgaaatactttgcagaaaaaataacttatgatgttccagaacaagatttagaagtggacttaaacagtatcccaggatacgttaagagtggctttgtaatcaaaaatgttcgcgaagttgtagacagaacataa

SEQ ID NO: 7 - protein, 560 aa, synthetic construct
  REGION 1..560: note = phaC
MTDKPAKGSTTLPATRMNVQNAILGLRGRDLLSTLRNVGRHGLRHPLHTAHHLLALGGQLGRVMLGDTPYQPNPRDARFSDPTWSQNPFYRRGLQAYLAWQKQTRQWIDESHLNDDDRARAHFLFNLINDALAPSNSLLNPLAVKELFNTGGQSLVRGVAHLLDDLRHNDGLPRQVDERAFEVGVNLAATPGAVVFRNELLELIQYSPMSEKQHARPLLVVPPQINKFYIFDLSATNSFVQYMLKSGLQVFMVSWRNPDPRHREWGLSSYVQALEEALNACRSISGNRDPNLMGACAGGLTMAALQGHLQAKKQLRRVRSATYLVSLLDSKFESPASLFADEQTIEAAKRRSYQRGVLDGGEVARIFAWMRPNDLIWNYWVNNYLLGKTPPAFDILYWNADSTRLPAALHGDLLEFFKLNPLTYASGLEVCGTPIDLQQVNIDSFTVAGSNDHITPWDAVYRSALLLGGERRFVLANSGHIQSIINPPGNPKAYYLANPKLSSDPRAWFHDAKRSEGSWWPLWLEWITARSGLLKAPRTELGNATYPPLGPAPGTYVLTR

SEQ ID NO: 8 - DNA, 1683 bp, synthetic construct
  misc_feature 1..1683: note = phaC
  source 1..1683: mol_type = other DNA
atgacagacaaaccggccaaaggatcgacaacgctccccgccacccgcatgaacgtgcagaacgccatcctcggcctgcgcggccgcgacctgctttccacgctgcgcaacgtcggccgccacggcctgcgccacccgctgcataccgcgcatcatctgctggcgcttggcgggcagttggggcgggtgatgctgggggacacgccctaccagccgaacccgcgtgacgcacgcttcagtgacccgacctggagccagaacccgttctaccgccgcggcctgcaagcctatctggcctggcagaagcagacacgccagtggatcgatgaaagccatttgaacgacgatgatcgagcccgcgcccacttcctgttcaacctgatcaacgatgcgctggcgcccagcaactcactgctcaatccgttggcggtcaaggagctgttcaacaccggcggccagagcctggtgcgcggcgtggctcacctgctcgacgacctgcgtcacaacgatgggctgcctcgtcaggtggacgagcgcgccttcgaagtgggcgttaacctggccgcaacccctggcgcagtggtatttcgcaacgagctgctggagctgatccagtactcgccgatgagcgaaaagcagcacgcacgcccactgctggtcgtgccgcctcagatcaacaagttctacatcttcgacctcagcgcgaccaacagcttcgtccagtacatgctcaaaagcggcttgcaggtgttcatggtcagctggcgcaaccccgacccacgccaccgtgaatggggcctttccagctatgtgcaagccctggaggaagcgctcaatgcctgccgcagtatcagcggcaaccgcgaccccaacctgatgggtgcctgtgccggcggcctgaccatggccgcactgcaaggccatctgcaagccaagaagcaattgcgccgggtgcgcagtgccacgtatctggtcagcttgctggacagcaagttcgaaagcccggccagcctgttcgccgatgagcagaccatcgaagcggccaagcgacgctcctatcagcgtggcgtgctggacggtggtgaagtggcgcggatcttcgcctggatgcggcccaacgacctgatctggaactactgggtaaacaactacctgctcggcaagacaccgccggcgttcgacatcctgtactggaatgccgacagcacgcgcctgcccgccgcgctgcatggcgacctgctggagtttttcaagctcaaccccttgacctacgcgtccgggctggaggtgtgcggtacgccgatcgacctgcagcaggtcaatatcgacagctttaccgtggccggcagcaacgaccacatcacaccatgggatgcggtgtaccgctcggccttgctgctgggtggcgagcggcgcttcgtgctggccaacagcgggcatatccagagcatcatcaacccgccaggcaaccccaaggcctactacctggccaaccccaagctgagcagcgacccacgcgcctggttccacgacgccaagcgcagtgaaggcagctggtggccgttgtggctggagtggatcaccgcacgctccggcctgctcaaggcaccgcgtactgaactgggcaacgccacttacccaccgctaggccccgcgccaggcacctacgtgctgacccgatga

SEQ ID NO: 9 - protein, 156 aa, synthetic construct
  REGION 1..156: note = phaJ
MSQVTNTPYEALEVGQKAEYKKSVEERDIQLFAAMSGDHNPVHLDAEFAAKSMFRERIAHGMFSGALISAAVACTLPGPGTIYLGQQMSFQKPVKIGDTLTVRLEILEKLPKFKVRIATNVYNQNDELVVAGEAEILAPRKQQTVELVSPPNFVAS

SEQ ID NO: 10 - DNA, 471 bp, synthetic construct
  misc_feature 1..471: note = phaJ
  source 1..471: mol_type = other DNA
atgtcccaggtcaccaacacgccttacgaagccctcgaagtcggccagaaggccgagtacaagaagtccgttgaagaacgcgacatccagctgttcgccgcgatgtccggtgaccacaacccggttcacctggatgccgagttcgccgccaagagcatgttccgcgaacgtattgcccatggcatgttcagcggcgcgctgatcagtgcggcagtggcctgcaccctgcctggccctggcaccatctacctgggccagcagatgagcttccagaaaccggtgaaaatcggcgacaccctgaccgtgcgcctggagatcctcgagaagctgcccaagttcaaggtgcgcatcgccaccaatgtgtacaaccagaacgacgagctggtggtcgcgggtgaagccgagatcctggcgccgcgcaagcagcagaccgtcgagctggtatcgccgccgaactttgtggcgagctga

SEQ ID NO: 11 - DNA, 28 bp, synthetic construct
  misc_feature 1..28: note = primer
  source 1..28: mol_type = other DNA
atgtatatctccttcttaattaagcatg

SEQ ID NO: 12 - DNA, 23 bp, synthetic construct
  misc_feature 1..23: note = primer
  source 1..23: mol_type = other DNA
tttcaccctaaattcgagagatt

SEQ ID NO: 13 - DNA, 48 bp, synthetic construct
  misc_feature 1..48: note = primer
  source 1..48: mol_type = other DNA
tacgccaagcttgcatgcctgcagccgaaagaggtccgcgagttcgca

SEQ ID NO: 14 - DNA, 20 bp, synthetic construct
  misc_feature 1..20: note = primer
  source 1..20: mol_type = other DNA
gggagactttctcgtgatgg

SEQ ID NO: 15 - DNA, 45 bp, synthetic construct
  misc_feature 1..45: note = primer
  source 1..45: mol_type = other DNA
ccatcacgagaaagtctccctaccgaaagcacaagcgtgagcgac

SEQ ID NO: 16 - DNA, 48 bp, synthetic construct
  misc_feature 1..48: note = primer
  source 1..48: mol_type = other DNA
attcgagctcggtacccggggatccatctcggtgatgcgccgcgacca

SEQ ID NO: 17 - DNA, 47 bp, synthetic construct
  misc_feature 1..47: note = primer
  source 1..47: mol_type = other DNA
tacgccaagcttgcatgcctgcagcaggtgccggtggtcaatcgtcc

SEQ ID NO: 18 - DNA, 24 bp, synthetic construct
  misc_feature 1..24: note = primer
  source 1..24: mol_type = other DNA
gcagtctccaagatcgacgtgttg

SEQ ID NO: 19 - DNA, 42 bp, synthetic construct
  misc_feature 1..42: note = primer
  source 1..42: mol_type = other DNA
acgtcgatcttggagactgcaagcggagcctccgatgacctt

SEQ ID NO: 20 - DNA, 47 bp, synthetic construct
  misc_feature 1..47: note = primer
  source 1..47: mol_type = other DNA
attcgagctcggtacccggggatccagtgcgtcgtaggtcatcgccc

SEQ ID NO: 21 - DNA, 48 bp, synthetic construct
  misc_feature 1..48: note = primer
  source 1..48: mol_type = other DNA
tacgccaagcttgcatgcctgcagacaccggcaacatggaattgctgc

SEQ ID NO: 22 - DNA, 25 bp, synthetic construct
  misc_feature 1..25: note = primer
  source 1..25: mol_type = other DNA
ccgcggacttctttcagtaccgggg

SEQ ID NO: 23 - DNA, 44 bp, synthetic construct
  misc_feature 1..44: note = primer
  source 1..44: mol_type = other DNA
gtactgaaagaagtccgcggagaccaccatgcccaccgattccg

SEQ ID NO: 24 - DNA, 47 bp, synthetic construct
  misc_feature 1..47: note = primer
  source 1..47: mol_type = other DNA
attcgagctcggtacccggggatccgattccgaagatcagcttccgg

SEQ ID NO: 25 - DNA, 48 bp, synthetic construct
  misc_feature 1..48: note = primer
  source 1..48: mol_type = other DNA
tacgccaagcttgcatgcctgcagtcactccacgctcacccaggccac

SEQ ID NO: 26 - DNA, 24 bp, synthetic construct
  misc_feature 1..24: note = primer
  source 1..24: mol_type = other DNA
cctctatgtcgggtgcagtgcggc

SEQ ID NO: 27 - DNA, 45 bp, synthetic construct
  misc_feature 1..45: note = primer
  source 1..45: mol_type = other DNA
cactgcacccgacatagaggggagccggacatgaccgacagttat

SEQ ID NO: 28 - DNA, 48 bp, synthetic construct
  misc_feature 1..48: note = primer
  source 1..48: mol_type = other DNA
attcgagctcggtacccggggatcctcatgtaatgcaggtcgctgtgg

SEQ ID NO: 29 - DNA, 46 bp, synthetic construct
  misc_feature 1..46: note = primer
  source 1..46: mol_type = other DNA
tacgccaagcttgcatgcctgcagctccagaacgtacgaaccgaac

SEQ ID NO: 30 - DNA, 23 bp, synthetic construct
  misc_feature 1..23: note = primer
  source 1..23: mol_type = other DNA
gaaaacctccgacactgaagata

SEQ ID NO: 31 - DNA, 41 bp, synthetic construct
  misc_feature 1..41: note = primer
  source 1..41: mol_type = other DNA
cttcagtgtcggaggttttcggctcgtgagtacttatcaac

SEQ ID NO: 32 - DNA, 47 bp, synthetic construct
  misc_feature 1..47: note = primer
  source 1..47: mol_type = other DNA
attcgagctcggtacccggggatccaccttgatggtgcccacaccgt

SEQ ID NO: 33 - DNA, 48 bp, synthetic construct
  misc_feature 1..48: note = primer
  source 1..48: mol_type = other DNA
tacgccaagcttgcatgcctgcagtgaacgcctgagtccgacagcacg

SEQ ID NO: 34 - DNA, 25 bp, synthetic construct
  misc_feature 1..25: note = primer
  source 1..25: mol_type = other DNA
gatgagtcctccgtcccggtgccgc

SEQ ID NO: 35 - DNA, 45 bp, synthetic construct
  misc_feature 1..45: note = primer
  source 1..45: mol_type = other DNA
accgggacggaggactcatcccgtcatgaccatcgaattcgagtc

SEQ ID NO: 36 - DNA, 50 bp, synthetic construct
  misc_feature 1..50: note = primer
  source 1..50: mol_type = other DNA
attcgagctcggtacccggggatcccgaggaggttggccaccggcacgcg

SEQ ID NO: 37 - DNA, 49 bp, synthetic construct
  misc_feature 1..49: note = primer
  source 1..49: mol_type = other DNA
tacgccaagcttgcatgcctgcagtcgccaccgtgaagtcgacgaggtt

SEQ ID NO: 38 - DNA, 45 bp, synthetic construct
  misc_feature 1..45: note = primer
  source 1..45: mol_type = other DNA
ttatttctgctagaaagcttgtcaggcctccggacttcccggatc

SEQ ID NO: 39 - DNA, 45 bp, synthetic construct
  misc_feature 1..45: note = primer
  source 1..45: mol_type = other DNA
ataagagaaagggagtccacatgcccttcttcgacggtgtcagga

SEQ ID NO: 40 - DNA, 50 bp, synthetic construct
  misc_feature 1..50: note = primer
  source 1..50: mol_type = other DNA
attcgagctcggtacccggggatccgccggtgaaagtcaggttgacggga

SEQ ID NO: 41 - DNA, 48 bp, synthetic construct
  misc_feature 1..48: note = primer
  source 1..48: mol_type = other DNA
tacgccaagcttgcatgcctgcaggggcagtccgccgagcgcctgcgg

SEQ ID NO: 42 - DNA, 45 bp, synthetic construct
  misc_feature 1..45: note = primer
  source 1..45: mol_type = other DNA
ttatttctgctagaaagctttctggctctgtcgcaccccgatggc

SEQ ID NO: 43 - DNA, 46 bp, synthetic construct
  misc_feature 1..46: note = primer
  source 1..46: mol_type = other DNA
ataagagaaagggagtccacatggcagccatgcccttcttcgacgg

SEQ ID NO: 44 - DNA, 49 bp, synthetic construct
  misc_feature 1..49: note = primer
  source 1..49: mol_type = other DNA
attcgagctcggtacccggggatcccggtgacggcaagccgttcatcgg

SEQ ID NO: 45 - DNA, 43 bp, synthetic construct
  misc_feature 1..43: note = primer
  source 1..43: mol_type = other DNA
tacgccaagcttgcatgcctgcagatgcggaacgggaccatgg

SEQ ID NO: 46 - DNA, 46 bp, synthetic construct
  misc_feature 1..46: note = primer
  source 1..46: mol_type = other DNA
ttatttctgctagaaagctttgcaccttctcaaactctctgtggac

SEQ ID NO: 47 - DNA, 43 bp, synthetic construct
  misc_feature 1..43: note = primer
  source 1..43: mol_type = other DNA
ataagagaaagggagtccacatgtgtcccgtccatacaccagc

SEQ ID NO: 48 - DNA, 43 bp, synthetic construct
  misc_feature 1..43: note = primer
  source 1..43: mol_type = other DNA
attcgagctcggtacccggggatccgacccgttcccagctcgg

SEQ ID NO: 49 - DNA, 44 bp, synthetic construct
  misc_feature 1..44: note = primer
  source 1..44: mol_type = other DNA
tacgccaagcttgcatgcctgcagctggtccatcgtctgggagg

SEQ ID NO: 50 - DNA, 39 bp, synthetic construct
  misc_feature 1..39: note = primer
  source 1..39: mol_type = other DNA
ttatttctgctagaaagcttcgcgaggatcaccacgtca

SEQ ID NO: 51 - DNA, 43 bp, synthetic construct
  misc_feature 1..43: note = primer
  source 1..43: mol_type = other DNA
ataagagaaagggagtccacatgtgcatgcgtggtcattctgt

SEQ ID NO: 52 - DNA, 43 bp, synthetic construct
  misc_feature 1..43: note = primer
  source 1..43: mol_type = other DNA
attcgagctcggtacccggggatccaggaaaccgacgccaccc

SEQ ID NO: 53 - DNA, 27 bp, synthetic construct
  misc_feature 1..27: note = synthetic RBS
  source 1..27: mol_type = other DNA
ctcgcagccggtggaaaggaggtctat

SEQ ID NO: 54 - DNA, 47 bp, synthetic construct
  misc_feature 1..47: note = primer
  source 1..47: mol_type = other DNA
atagacctcctttccaccggctgcgagattaagcacctggaggtggt

SEQ ID NO: 55 - DNA, 21 bp, synthetic construct
  misc_feature 1..21: note = primer
  source 1..21: mol_type = other DNA
ttcttcgcaaaaatcgtcccc

SEQ ID NO: 56 - DNA, 114 bp, synthetic construct
  misc_feature 1..114: note = promoter and non-translation sequence
  source 1..114: mol_type = other DNA
tgtgcgggctctaacacgtcctagtatggtaggatgagcaacatttcgacgccgagagattcgccgcccgaaatgagcacgatccgcatgcttaattaagaaggagatatacat

SEQ ID NO: 57 - DNA, 22 bp, synthetic construct
  misc_feature 1..22: note = primer
  source 1..22: mol_type = other DNA
ctgaatggcgggagtatgaaaa

SEQ ID NO: 58 - DNA, 21 bp, synthetic construct
  misc_feature 1..21: note = primer
  source 1..21: mol_type = other DNA
tacaacgtcgtgactgggaaa

SEQ ID NO: 59 - DNA, 44 bp, synthetic construct
  misc_feature 1..44: note = primer
  source 1..44: mol_type = other DNA
tacgccaagcttgcatgcctgcagctgtggatctggtgcacctc

SEQ ID NO: 60 - DNA, 39 bp, synthetic construct
  misc_feature 1..39: note = primer
  source 1..39: mol_type = other DNA
ttatttctgctagaaagcttgatccgaacctcctcgtcc

SEQ ID NO: 61 - DNA, 41 bp, synthetic construct
  misc_feature 1..41: note = primer
  source 1..41: mol_type = other DNA
ataagagaaagggagtccacatgtggccgcctaccagtgcc

SEQ ID NO: 62 - DNA, 44 bp, synthetic construct
  misc_feature 1..44: note = primer
  source 1..44: mol_type = other DNA
attcgagctcggtacccggggatccatagggcggtgcgggttcg

SEQ ID NO: 63 - DNA, 114 bp, synthetic construct; identical to SEQ ID NO: 56
  misc_feature 1..114: note = promoter and non-translation sequence
  source 1..114: mol_type = other DNA

(19) United States
(12) Patent Application Publication
(10) Pub. No.: US 2025/0171814 A1
(43) Pub. Date: May 29, 2025

(54) NOVEL LIPID PEROXIDATION SYSTEM AND METHOD FOR PREPARING BIOFUEL AND BIOPOLYMER USING SAME

(71) Applicant: KOREA ADVANCED INSTITUTE OF SCIENCE AND TECHNOLOGY, Daejeon (KR)

(72) Inventors: Sang Yup LEE, Daejeon (KR); In Jin CHO, Daejeon (KR)

(21) Appl. No.: 18/837,131

(22) PCT Filed: Feb. 10, 2023
(86) PCT No.: PCT/KR2023/001999

§ 371 Date: Aug. 8, 2024

(30) Foreign Application Priority Data
Feb. 11, 2022 (KR) 10-2022-0018142

(51) Int. Cl. C12P 7/6409; C12N 1/20; C12N 9/02; C12N 9/08; C12N 9/10; C12P 5/02; C12R 1/15; C12R 1/32

(52) U.S. Cl. CPC C12P 7/6409; C12N 1/20; C12N 9/0061; C12N 9/0065; C12N 9/1029; C12P 5/02; C12Y 110/03002; C12Y 111/01014; C12Y 203/01203; C12N 2510/02; C12R 2001/15; C12R 2001/32

(57) ABSTRACT

The present invention relates to recombinant bacteria to which a novel lipid peroxidation system has been applied and a method for preparing a biofuel and a biopolymer using same. The artificial lipid peroxidation system of the present invention can be used to prepare ultra-high concentrations of free fatty acids through artificial lipid peroxidation without cell death, and a biofuel and a biopolymer can be prepared therefrom. In addition, the artificial lipid peroxidation system according to the present invention increases intracellular redox energy density and can thus additionally promote carbon dioxide fixation. Accordingly, it is possible to prepare increased quantities of a biofuel and a biopolymer.

### TECHNICAL FIELD

The present invention relates to a recombinant bacterium into which a novel lipid peroxidation system has been introduced and methods of producing biofuels and biopolymers using the same, and more specifically, to a recombinant bacterium with induced cell membrane lipid peroxidation ability, characterized in that a heterologous peroxidase-encoding gene is introduced thereinto, and methods of producing free fatty acids, biofuels or biopolymers using the same.

### BACKGROUND ART

Free fatty acids are the most basic monomers in petrochemical refinery systems, and various types of fuels, polymers, and other compounds can be produced through these free fatty acids. While there is a growing desire to reorganize into an environmentally friendly refinery system, existing bio-based refineries have limitations in that they use edible resources to produce value-added compounds, and non-edible biomass-based refinery platforms of microorganisms have limitations in that the production of value-added compounds is low.

In an attempt to convert petrochemical refinery systems, which accelerate global warming and environmental pollution, into biological refinery systems, studies on overproducing triacylglycerols from glucose, an non-edible biomass, and overproducing free fatty acids by hydrolysis of the fat, were conducted through metabolic engineering studies on R. opacus PD630 (Kim et al., Nature Chem. Biol. 15, 721-729, 2019). However, this biological method requires a long culture time because it must wait for over-accumulation of intracellular triacylglycerols. In addition, this method has the limitation of being less competitive than existing petrochemical methods due to a limited proportion of triacylglycerols that can be accumulated within cells.

In general, lipid peroxidation leads to cell lysis and death (Hong et al., Proc Natl Acad Sci USA 16 (20) 10064-10071, 2019). However, if cell lysis and cell death in fed-batch culture continue, biomass does not accumulate much, and thus it is difficult to produce a large amount of a desired product.

Accordingly, the present inventors have made extensive efforts to develop technology that decomposes cell membrane lipids without cell lysis, and as a result, have found that, when a heterologous peroxidase-encoding gene is introduced into bacteria, the bacteria can overproduce free fatty acids while the thickness of the cell membrane of the bacteria is reduced without cell lysis, thereby completing the present invention.

### PRIOR ART DOCUMENTS

### Non-Patent Documents

(Non-Patent Document 1) 1. Kim, H. M. et al., Nature Chem. Biol., 15, 721-729, 2019 (https://doi.org/10.1038/s41589-019-0295-5)
(Non-Patent Document 2) 2. Hong et al., Proc Natl Acad Sci USA 16 (20) 10064-10071, 2019

### SUMMARY OF THE INVENTION

Therefore, an object of the present invention is to provide a recombinant bacterium having induced cell membrane lipid peroxidation capability to degrade cell membrane lipids without cell lysis and cell death.

Another object of the present invention is to provide a method of producing free fatty acids using the recombinant bacterium having induced cell membrane lipid peroxidation capability.

Still another object of the present invention is to provide a method of producing hydrocarbons using the recombinant bacterium having induced cell membrane lipid peroxidation capability.

Yet another object of the present invention is to provide a method of producing polyolefin using the recombinant bacterium having induced cell membrane lipid peroxidation capability.

Still yet another object of the present invention is to provide a method of producing polyhydroxyalkanoates using the recombinant bacterium with induced cell membrane lipid peroxidation ability.

A further object of the present invention is to provide a method of producing vinyl polymers including vinyl-copolymerized polyhydroxyalkanoates using the recombinant bacterium with induced cell membrane lipid peroxidation capability.

Another further object of the present invention is to provide a method of fixing carbon dioxide using the recombinant bacterium induced cell membrane lipid peroxidation capability.

To achieve the above objectives, the present invention provides a recombinant bacterium with induced cell membrane lipid peroxidation capability, characterized in that a heterologous peroxidase-encoding gene is introduced thereinto.

The present invention also provides a method of producing free fatty acids by cell membrane lipid peroxidation comprising the steps of:
(a) culturing the recombinant bacterium to produce free fatty acids by cell membrane lipid peroxidation; and
(b) recovering the synthesized free fatty acids.

The present invention also provides a method for producing hydrocarbons comprising the steps of:
(a) culturing the recombinant bacterium to produce hydrocarbons; and
(b) recovering the produced hydrocarbons.

The present invention also provides a method for producing polyolefin comprising the steps of:
(a) culturing the recombinant bacterium to produce polyolefin; and
(b) recovering the produced polyolefin.

The present invention also provides a method for producing polyhydroxyalkanoates comprising the steps of:
(a) culturing the recombinant bacterium to produce polyhydroxyalkanoates; and
(b) recovering the produced polyhydroxyalkanoates.

The present invention also provides a method for producing vinyl polymers including vinyl-copolymerized polyhydroxyalkanoates comprising the steps:
(a) culturing the recombinant bacterium to produce vinyl polymers including vinyl-copolymerized polyhydroxyalkanoates; and
(b) recovering the produced vinyl polymers including vinyl-copolymerized polyhydroxyalkanoates.

The present invention also provides recombinant Rhodococcus opacus with induced cell membrane lipid peroxidation ability, characterized in that a lignin peroxidase-encoding gene is introduced thereinto.

The present invention also provides a method of producing free fatty acids by cell membrane lipid peroxidation comprising the steps of:
(a) culturing the recombinant Rhodococcus opacus to produce free fatty acids by cell membrane lipid peroxidation; and
(b) recovering the produced free fatty acids.

The present invention also provides a method of producing triacylglycerols by cell membrane lipid peroxidation comprising the steps of:
a) culturing the recombinant bacterium to overproduce triacylglycerols by cell membrane lipid peroxidation; and
(b) recovering the produced triacylglycerols.

The present invention also provides a method for producing hydrocarbons comprising the steps of:
(a) cultivating the recombinant Rhodococcus opacus to produce hydrocarbons; and
(b) recovering the produced hydrocarbons.

The present invention also provides a method for producing polyolefin comprising the steps of:
(a) culturing the recombinant Rhodococcus opacus to produce polyolefin; and
(b) recovering the produced polyolefin.

The present invention also provides a method for producing polyhydroxyalkanoates comprising the steps of:
(a) culturing the recombinant Rhodococcus opacus to produce polyhydroxyalkanoates; and
(b) recovering the produced polyhydroxyalkanoates.

The present invention also provides a method for producing vinyl polymers including vinyl-copolymerized polyhydroxyalkanoates comprising the steps of:
(a) culturing the recombinant Rhodococcus opacus to produce vinyl polymers including vinyl-copolymerized polyhydroxyalkanoates; and
(b) recovering the produced vinyl polymers including vinyl-copolymerized polyhydroxyalkanoates.

### BRIEF DESCRIPTION OF DRAWINGS

FIG. 1 shows the results of analyzing the production of free fatty acids in a ROA PCO strain, a R. opacus strain into which the lignin peroxidase gene from Phanerochaete carnosa has been introduced.

FIG. 2 shows the results of analyzing the production of free fatty acids by performing an in vitro assay for each strain in order to confirm whether artificial lipid peroxidation by the lignin peroxidase gene from Phanerochaete carnosa would occur in Corynebacterium glutamicum, Rhodococcus opacus, and Rhodococcus ruber, which are representative Gram-positive bacteria.

FIG. 3 shows the results of observing, by ultrathin sectioning TEM, the change in the cell membrane thickness of the recombinant ROA PCO strain to which artificial lipid peroxidation has been applied (A to C), and a box-and-whisker graph (D) quantifying the change in the cell membrane thickness.

FIG. 4 shows the results of analyzing, by TLC, changes in the mycomembrane layer of the recombinant ROA PCO strain. (A) wild-type Rhodococcus, (B) FFA PCO, (C) FFA PCO AFLO, (D) Mycobacterium bovis-derived trehalose 6,6′ standard, and (E) Mycobacterium tuberculosis-derived mycolic acid standard.

FIG. 5 shows the tendency of changes in oxidation-reduction potential of wild-type Rhodococcus and recombinant FFA PCO strains under fed-batch culture conditions through oxidation-reduction potential (ORP) measurement.

FIG. 6A shows the results of observing, by TEM, the interior of the cells of the recombinant FFA PCO strain under initial fed-batch culture conditions, and FIG. 6B shows the amount of triacylglycerols produced in the cells of the recombinant FFA PCO strain under initial fed-batch culture and the content of triacylglycerols per dry cell weight.

FIG. 7 shows the results of examining the changes in dry cell weight of the recombinant FFA PCO strain and FFA PCO AFLO strain in fed-batch culture.

FIG. 8 shows the tendency of changes in the oxidation-reduction potential of wild-type Rhodococcus, the recombinant FFA PCO strain, and the recombinant FFA PCO AFLO strain under fed-batch culture conditions through ORP measurement.

FIG. 9 shows the free fatty acid production profile of the FFA PCO AFLO strain in fed-batch culture.

FIG. 10 shows the results of glucose adaptive evolution of the FFA PCO strain. Comparison of population sizes on solid media: A: FFA PCO (control), B: glucose-adaptive evolved FFA PCO (FG FFA PCO).

FIG. 11 shows the fed-batch culture profile of FG FFA PCO, a glucose-adaptive evolved strain.

FIG. 12 shows a comparison between hydrocarbon production fermentation profiles in fed-batch culture.

FIG. 13 shows the results of examining the hydrocarbon titer of the FG FFA PCO strain (A) and the FG FFA PCO AFLO strain (B) in fed-batch culture.

FIG. 14 shows the increase in biomass versus the glucose consumption of the FG FFA PCO strain by the additional supplementation of carbon dioxide during the fed-batch culture.

FIG. 15 shows the results of comparing the carbon dioxide fixation and “hydrocarbon production from glucose” profile of the FG FFA PCO strain when carbon dioxide was additionally added (CFGFFAPCO) with the carbon dioxide fixation and “hydrocarbon production from glucose” profile of the FG FFA PCO strain when no carbon dioxide was added (FGFFAPCO).

FIG. 16 shows the system metabolic engineering manipulation of recombinant Rhodococcus for the production of high concentrations of hydrocarbons and the results thereof. Specifically, FIG. 16A shows the hydrocarbon production pathway manipulation pathway for intracellular hydrocarbon accumulation, FIG. 16B shows the fed-batch culture profile of the FG FFA PCO AFLO OLET strain into which the oleT gene has been introduced, and FIG. 16C shows the fed-batch culture profile of the FG FFAdA PCO AFLO OLET strain with the alkB gene deleted.

FIG. 17 shows the results of fed-batch culture of cells to which carbon dioxide and formic acid as sole carbon and energy sources have been added, and shows the results of comparing the dissolved oxygen level change profiles of the wild-type R. opacus strain and the recombinant FFA PCO strain.

FIG. 18 shows the results of comparing the carbon dioxide fixation and “hydrocarbon production from glucose” profile of the FG FFAdA PCO AFLO OLET strain when carbon dioxide was additionally added (CFGFFAdAPAO) with the carbon dioxide fixation and “hydrocarbon production from glucose” profile of the FG FFAdA PCO AFLO OLET strain when no carbon dioxide was added (FGFFAdAPAO).

FIG. 19 depicts a photograph showing crystallization during solvent extraction of a new polyolefin produced by recombinant Rhodococcus (A) and a photograph showing polyolefin precipitation by an acetone-methanol solubility difference after solvent extraction (B).

FIG. 20 shows the results of identifying the polymer structure by comparing ¹H NMR data of a new polymer produced by recombinant Rhodococcus (A) with ¹H NMR data of saturated MCL-PHAs from Pseudomonas (B) and ¹H NMR data of unsaturated MCL-PHAs (C).

FIG. 21 shows the results of identifying the polymer structure by comparing ¹³C NMR data of a new polymer produced by recombinant Rhodococcus (A) with ¹³C NMR data of saturated MCL-PHAs from Pseudomonas (B) and ¹³C NMR data of unsaturated MCL-PHAs (C).

FIG. 22 shows the results of identifying the polymer structure by comparing ¹H-¹³C HSQC NMR data of a new polymer produced by recombinant Rhodococcus (A and B) with ¹H-¹³C HSQC NMR data of saturated MCL-PHAs from Pseudomonas (C) and ¹H-¹³C HSQC NMR data of saturated MCL-PHAS (D).

FIG. 23 shows the results of producing a new vinyl-copolymerized copolymer (a vinyl-copolymerized polyhydroxyalkanoate) by the radical system of recombinant R. opacus PD630 based on artificial lipid peroxidation. Specifically, FIG. 23A shows the design of a recombinant Rhodococcus system for producing a new polymer, FIG. 23B depicts TEM photographs showing the interior of the polymer-producing Rhodococcus system at 137 hours of fed-batch culture, and FIG. 23C shows the results of XPS analysis performed to determine whether radical polymerization on the branch of the new polymer occurred.

FIG. 24 shows the results of comparing the molecular weights of new vinyl-copolymerized polymers (vinyl-copolymerized polyhydroxyalkanoates) produced by the radical system of recombinant R. opacus PD630 based on artificial lipid peroxidation. FIG. 24A shows the molecular weight of the new polymer extracted from a culture of the FFAdA PCO AFLO OLET strain into which the polyhydroxyalkanoate production enzyme was not introduced. FIG. 24B shows the molecular weight of the new polymer extracted from a mixed culture of a recombinant FFA PCO strain that overproduces free fatty acids and a recombinant PPU PhaAB strain obtained by introducing the phaA and phaB genes into Pseudomonas with the ability to produce naturally occurring polyhydroxyalkanoates. FIG. 24C shows the molecular weight of the new polymer extracted from a culture of the recombinant ROA JC PCO strain obtained by introducing an artificial lipid peroxidation system and Pseudomonas-derived PhaJ and PhaC enzymes for polyhydroxyalkanoate production into wild-type Rhodococcus. FIG. 24D shows the molecular weight of the new polymer extracted from a culture (at 120 hours of culture) of a recombinant FFA JC PCO strain obtained by introducing Pseudomonas-derived PhaJ and PhaC enzymes for polyhydroxyalkanoate production into a recombinant FFA PCO strain that overproduces free fatty acids. FIG. 24E shows the molecular weight of the new polymer extracted from a culture (at 144 hours of culture) of a recombinant FFA JC PCO strain obtained by introducing Pseudomonas-derived PhaJ and PhaC enzymes for polyhydroxyalkanoate production into a recombinant FFA PCO strain that overproduces free fatty acids.

### DETAILED DESCRIPTION AND PREFERRED EMBODIMENTS OF THE INVENTION

### Detailed Description and Preferred Embodiments of the Invention

Unless otherwise defined, all technical and scientific terms used in the present specification have the same meanings as commonly understood by those skilled in the art to which the present invention pertains. In general, the nomenclature used in the present specification is well known and commonly used in the art.

Cells ubiquitously biosynthesize fatty acids for cell membrane synthesis. In the present invention, assuming that the conversion of cell membrane biosynthetic metabolism to free fatty acid metabolism could make it possible to produce large amounts of free fatty acids, heterologous peroxidase was used to induce cell membrane lipid peroxidation without cell death.

A method for highly efficient production of free fatty acids, which are the base compounds of the refinery, and free fatty acid-based value-added compounds, was found through a metabolic engineering study on Rhodococcus opacus PD630, a gram-positive bacterium that can produce triacylglycerols, a type of fat, through its own metabolic pathway.

In the present invention, based on the existing system of bacteria that degrades the inner membrane of the cell by lipid peroxidation, leading to selective death, it has been found that, when the outer membrane of the cell is degraded, it is possible to obtain large amounts of free fatty acids from the cell membrane and also to prevent cell death to enhance cell viability, thereby producing large amounts of biofuels and biopolymers, which are free fatty acid-based value-added compounds.

Therefore, the present invention is directed to a recombinant bacterium with induced cell membrane lipid peroxidation ability, characterized in that a heterogeneous peroxidase-encoding gene is introduced thereinto.

Lipid peroxidation generally leads to cell lysis and death (Hong et al., Proc Natl Acad Sci USA 16 (20) 10064-10071, 2019). However, if cell lysis and cell death in fed-batch culture continue, biomass does not accumulate much, and thus it is difficult to produce a large amount of a desired product. Therefore, in this study, technology for degrading cell membranes without cell lysis was developed. The present inventors have discovered a new enzyme that can be used to reduce the cell membrane thickness without cell lysis. This enzyme is not only a new discovery that has never been used before in the cell membrane lipidation process and but also is a key strategy for overproducing free fatty acids.

In the present invention, lipid peroxidation is intentionally induced by exogenous peroxidase and oxidizes the cell membrane, causing excessive accumulation of cellular fatty acids and extracellular free fatty acids. Furthermore, the present inventors hypothesized that the use of peroxidase would allow the cell membrane thickness of the engineered strain to decrease due to lipid peroxidation.

In one example of the present invention, a fungal peroxidase called lignin peroxidase (EC 1.11.1.14) from Phanerochaete carnosa was introduced into the Rhodococcus opacus (R. opacus) PD630 strain, thereby differentiating lipid peroxidation in the strain from existing intracellular native lipid peroxidation. Bacterial peroxidase has high specificity for native lipid peroxidation and thus can cause premature cell death, which is the purpose of existing lipid peroxidation. This enzyme is catalyzed by both phenolic and non-phenolic components after extracellular secretion. In order to confirm the possibility of extracellular secretion of the exogenous peroxidase after introduction into R. opacus, the signal peptide was classified. A recombinant strain obtained by introducing the lignin peroxidase from Phanerochaete carnosa into wild-type R. opacus was named ROA PCO, and it was confirmed that the fatty acid metabolic pathway for biomass production, especially cell membrane production, in the recombinant strain, was successfully changed to an extracellular free fatty acid metabolic pathway.

In the present invention, the peroxidase may be an enzyme having an EC number of EC 1.11.1.14.

The recombinant bacterium of the present invention may be a Gram-positive bacterium. Preferably, the recombinant bacterium may be a bacterium having mycolic acid as a cell membrane component, among Gram-positive bacteria, including Rhodococcus, Corynebacterium, Mycobacterium, Gordonia, Lawsonella, etc., without being limited thereto.

The recombinant bacterium of the present invention may be characterized in that an acyl-CoA synthetase-encoding gene is deleted and in that the promoter of a monoacylglycerol (MAG) lipase-encoding gene is replaced with an inducible promoter.

In the present invention, the recombinant bacterium may be characterized in that a 4-dichlorophenol 6-monooxygenase-encoding gene or a 2-dehydropantoate 2-reductase-encoding gene is additionally deleted.

The recombinant bacteria of the present invention may be characterized in that a laccase-like multicopper oxidase (LMCO)-encoding gene is additionally introduced.

The recombinant bacterium of the present invention may be an improved strain obtained through glucose adaptive evolution.

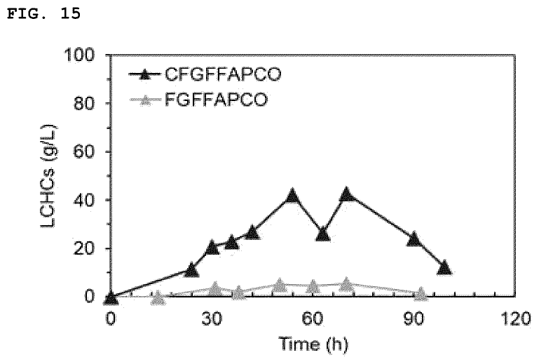

The recombinant bacterium of the present invention may be an improved strain that may use carbon dioxide as an additional carbon source (see FIG. 14).

The recombinant bacterium of the present invention may be characterized in that a gene encoding an enzyme that converts fatty acids into 1-alkenes is additionally introduced and in that, a gene encoding an enzyme that degrades hydrocarbons into 1-alcohols is deleted.

The recombinant bacterium of the present invention may be characterized in that a gene encoding a medium-chain-length (MCL) polyhydroxyalkanoate synthase and a gene encoding (R)-specific enoyl-CoA hydratase are additionally introduced for polyhydroxyalkanoate production.

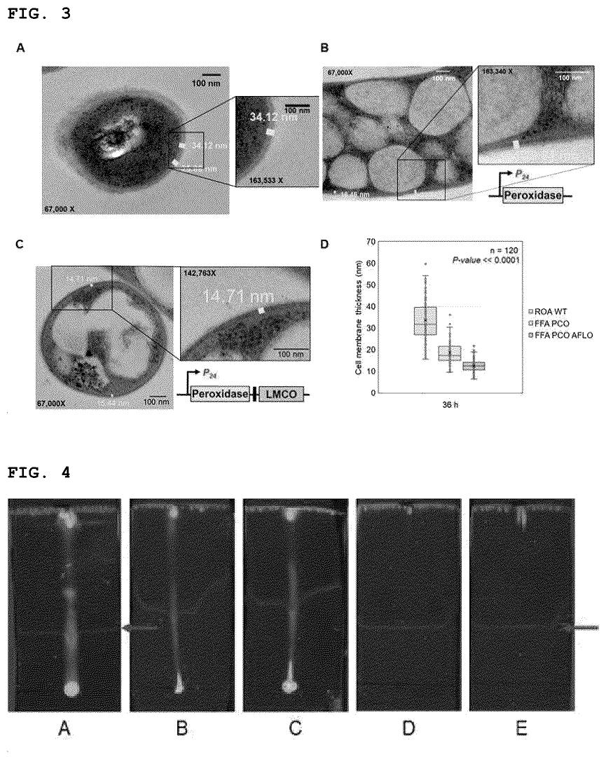

In one example of the present invention, it was confirmed that the cell membrane thickness of the ROA PCO strain decreased due to lipid peroxidation because the peroxidase secreted from the recombinant ROA PCO strain reacted with the cell membrane, centered on the cell membrane outer membrane composed mainly of mycolic acid. In addition, it was confirmed by TEM observation that the strain into which peroxidase was introduced had a larger cell size than the cell size of the wild-type strain (FIG. 3).

The recombinant bacterium of the present invention may have a reduced cell membrane thickness compared to the parent strain.

In one example of the present invention, it was confirmed that free fatty acids were produced at high concentrations by fed-batch culture of the recombinant bacterium.

In another aspect, the present invention is directed to a method of producing free fatty acids by cell membrane lipid peroxidation comprising the steps of:
(a) culturing the recombinant bacterium to produce free fatty acids by cell membrane lipid peroxidation; and
(b) recovering the produced free fatty acids.

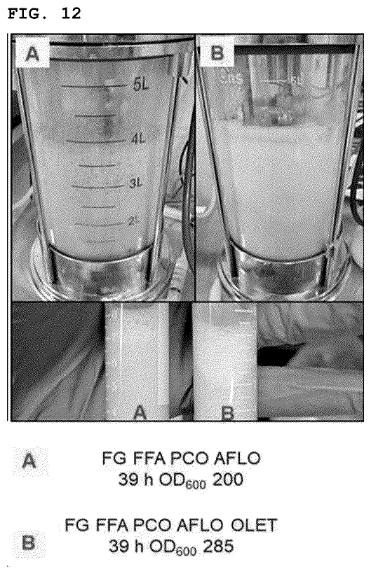

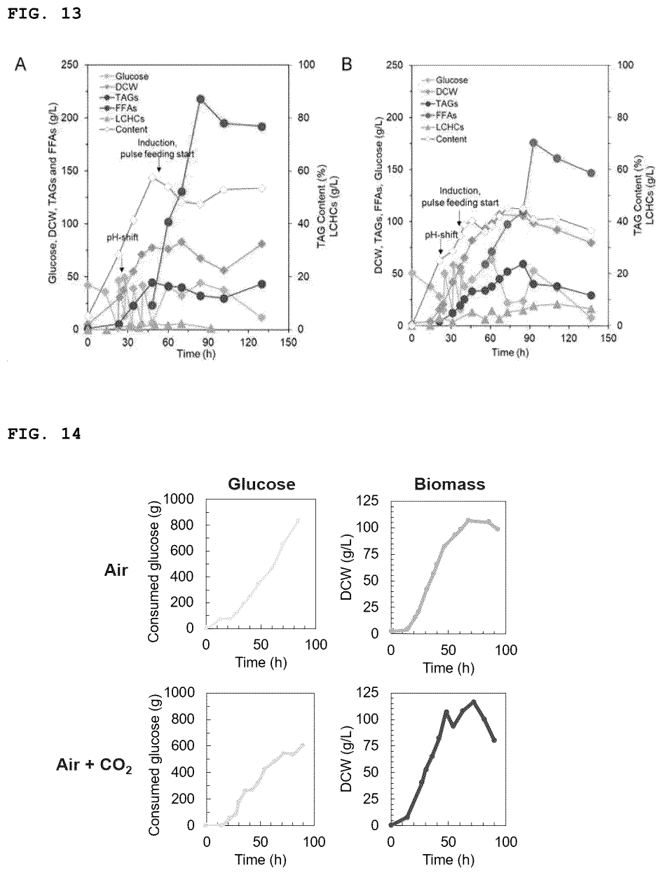

In one example of the present invention, peroxidase was used to induce artificial lipid peroxidation and increase the production of FFAs. Incidentally, it was confirmed that, since peroxidase could catalyze the alkane/alkene biosynthesis reaction, the recombinant strain into which peroxidase has been introduced produced hydrocarbons through fed-batch culture thereof (see FIGS. 12 and 13).

In another aspect, the present invention is directed to a method for producing hydrocarbons comprising the steps of:
(a) culturing the recombinant bacterium to produce hydrocarbons; and
(b) recovering the produced hydrocarbons.

In the present invention, step (a) may comprise additionally supplementing carbon dioxide.

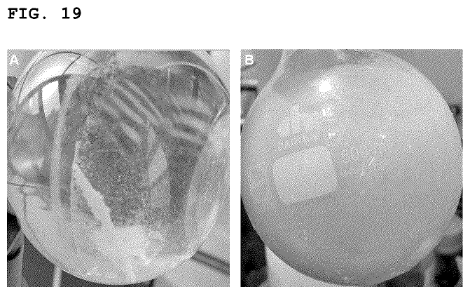

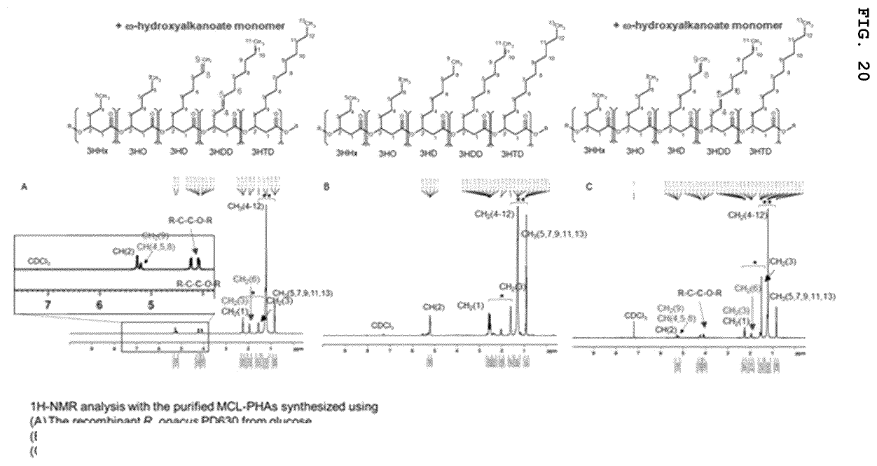

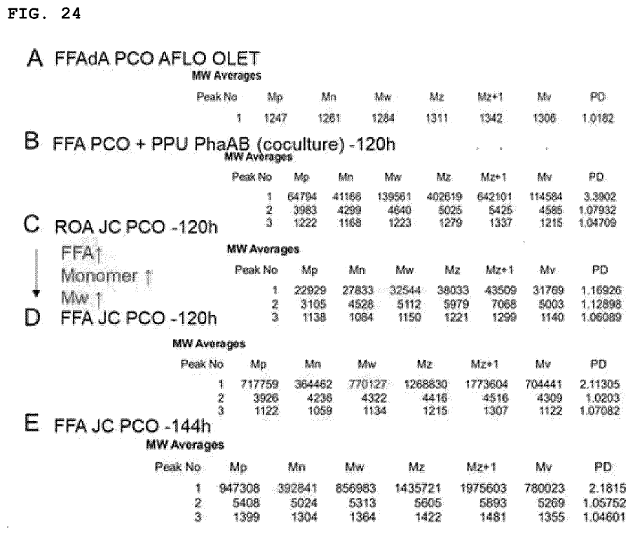

In one example of the present invention, peroxidase was used to induce artificial lipid peroxidation and increase the production of FFAs. Incidentally, it was confirmed that the oxidation-reduction energy that was increased by artificial lipid peroxidation produced polyolefins through the polymerization of alkenes and other unsaturated fatty acid derivatives (see FIGS. 19 and 24A).

In another aspect, the present invention is directed to a method for producing polyolefin comprising the steps of:
(a) culturing the recombinant bacterium to produce polyolefins; and
(b) recovering the produced polyolefins

In the present invention, step (a) may comprise additionally supplementing carbon dioxide.

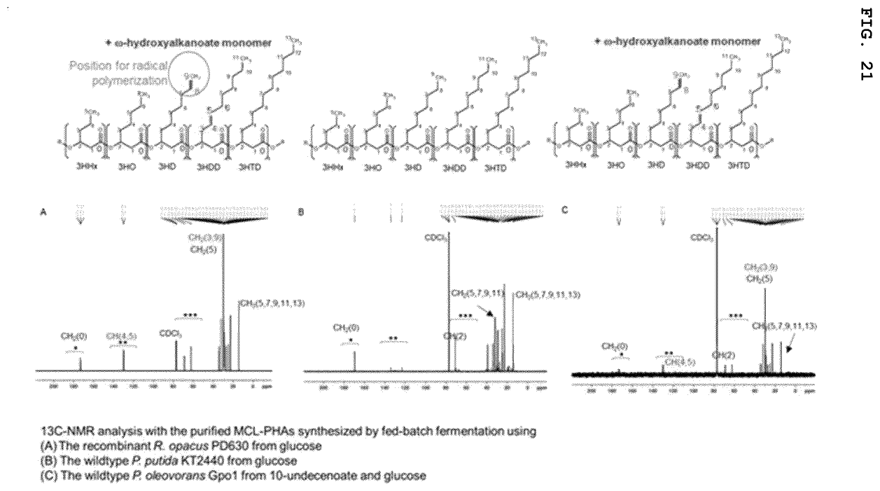

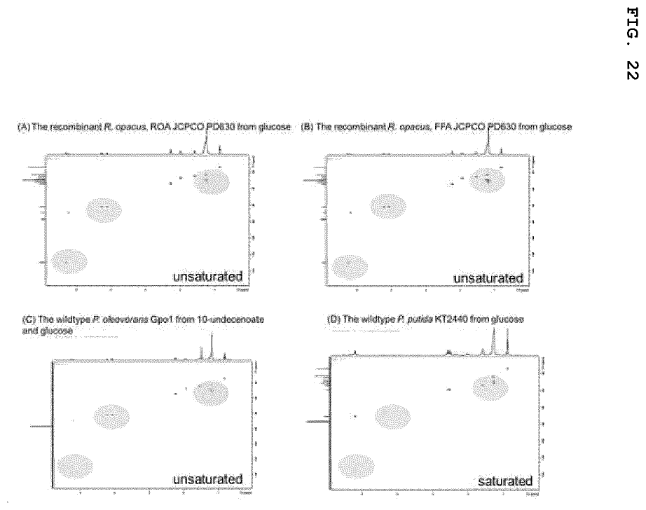

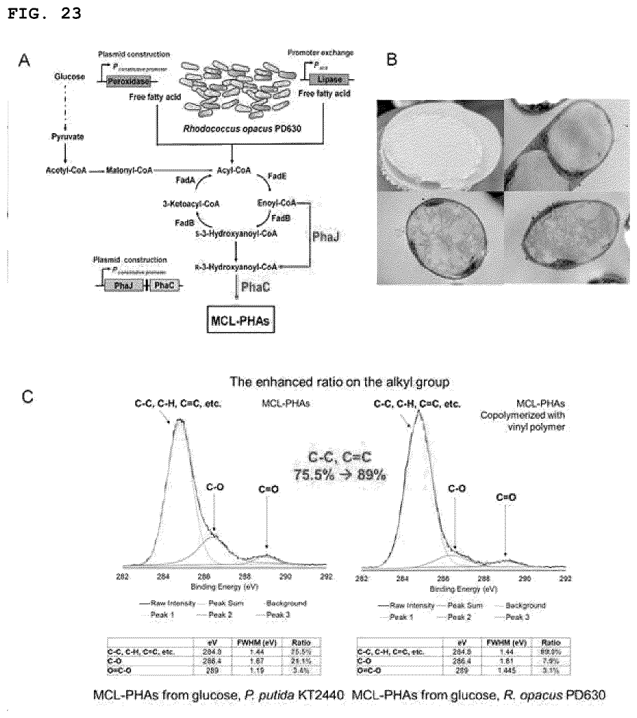

In one example of the present invention, for the production of medium-chain-length polyhydroxyalkanoates (MCL), a recombinant Rhodococcus opacus containing the medium-chain-length polyhydroxyalkanoate synthase encoded by phaC gene from Pseudomonas and the (R)-specific enoyl-CoA hydratase encoded by phaJ gene was constructed, and successful production of medium-chain-length polyhydroxyalkanoates by fed-batch culture of the recombinant strain was confirmed (see FIGS. 20 to 24).

In another aspect, the present invention is directed to a method for producing polyhydroxyalkanoates comprising the steps of:
(a) culturing the recombinant bacterium to produce polyhydroxyalkanoates; and
(b) recovering the produced polyhydroxyalkanoates.

In one example of the present invention, it was determined that a vinyl polymer could be synthesized through radical polymerization of carbon-carbon double-bonded alkenes containing monomers produced by artificial lipid peroxidation, and production of vinyl polymers including vinyl-copolymerized polyhydroxyalkanoates by the recombinant strains FFA JC PCO and FG FFA JCPCO was confirmed (see FIG. 24).

In another aspect, the present invention is directed to a method for producing vinyl polymers including vinyl-copolymerized polyhydroxyalkanoates comprising the steps of:
(a) culturing the recombinant bacterium to produce vinyl polymers including vinyl-copolymerized polyhydroxyalkanoates; and
(b) recovering the produced vinyl polymers including vinyl-copolymerized polyhydroxyalkanoates.

The vinyl polymers including vinyl-copolymerized polyhydroxyalkanoates in the present invention refer to a polymer produced by free radical polymerization of monomers using an in vivo artificial lipid peroxidation system as a radical initiator, without being limited thereto.

vinyl polymers including vinyl-copolymerized polyhydroxyalkanoates in the present invention is a new vinyl-copolymerized polymer obtained by polymerization between a polymer of a vinyl-based monomer having a carbon-carbon double bond and the polyester-based polymer including polyhydroxyalkanoates.

In another aspect, the present invention is directed to recombinant Rhodococcus opacus with induced cell membrane lipid peroxidation ability, characterized in that a lignin peroxidase-encoding gene is introduced therein.

In the present invention, the lignin peroxidase may have the amino acid sequence of SEQ ID NO: 1, and the lignin peroxidase-encoding gene may have the nucleotide sequence of SEQ ID NO: 2.

In the present invention, the recombinant Rhodococcus opacus may be characterized in that an acyl-CoA synthetase-encoding fadD gene is deleted.

In the present invention, the recombinant Rhodococcus opacus may be characterized in that the promoter of the LPD01036 gene or LPD02672 gene, which is a monoacylglycerol (MAG) lipase-encoding gene, is replaced with an inducible promoter.

In the present invention, the recombinant Rhodococcus opacus may be characterized in that LPD12046, a 4-dichlorophenol 6-monooxygenase-encoding gene, or LPD16168, a 2-dehydropantoate 2-reductase-encoding gene, is additionally deleted.

In the present invention, the recombinant Rhodococcus opacus may be characterized in that a laccase-like multicopper oxidase (LMCO)-encoding gene is additionally introduced, wherein the laccase-like multicopper oxidase (LMCO) has the amino acid sequence of SEQ ID NO: 3, and the LMCO-encoding gene has the nucleotide sequence of SEQ ID NO: 4.

The recombinant Rhodococcus opacus of the present invention may be an improved strain obtained through additional glucose adaptive evolution.

The recombinant bacterium of the present invention may be an improved strain that may use carbon dioxide as an additional carbon source (see FIG. 14).

Glucose adaptive evolution in the present invention means changing the aspect of the strain by periodically growing the strain on a solid medium containing glucose, in order to increase the utilization efficiency of glucose as a carbon source and energy source for the recombinant strain and lower the utilization efficiency of free fatty acids as a carbon source and an energy source, thereby relatively reducing free fatty acid consumption and increasing free fatty acid accumulation.

In the present invention, the recombinant Rhodococcus opacus may produce fatty acids having a carbon length of 8 to 22 carbon atoms, without being limited thereto.

In the present invention, the recombinant Rhodococcus opacus may be characterized in that a gene encoding an OleT enzyme that converts fatty acids into 1-alkenes is additionally introduced, wherein the OleT enzyme may have the amino acid sequence of SEQ ID NO: 5, and the gene encoding the OleT enzyme may have the nucleotide sequence of SEQ ID NO: 6.

In the present invention, recombinant the Rhodococcus opacus may be characterized in that alkB, a gene encoding an enzyme that degrades hydrocarbons into 1-alcohols, is deleted.

In the present invention, the recombinant Rhodococcus opacus may be characterized in that it can be confirmed that a polyolefin with a molecular weight of 1,000 or more is produced in the supernatant without additional reaction by causing intracellular radical polymerization (FIGS. 19 and 24).

In the present invention, the recombinant Rhodococcus opacus may be characterized in that phaC, a medium-chain-length (MCL) polyhydroxyalkanoate synthase-encoding gene, and phaJ, an (R)-specific enoyl-CoA hydratase-encoding gene, are introduced.

The phaC may have a nucleotide sequence encoding the amino acid sequence of SEQ ID NO: 7, and the phaC may have the nucleotide sequence of SEQ ID NO: 8.

In the present invention, the phaJ may have a nucleotide sequence encoding the amino acid sequence of SEQ ID NO: 9, and the phaJ may have the nucleotide sequence of SEQ ID NO: 10.

In one example of the present invention, it was confirmed that the cell membrane thickness of the ROA PCO strain decreased due to lipid peroxidation because the peroxidase secreted from the recombinant ROA PCO strain reacted with the cell membrane, centered on the cell membrane outer membrane composed mainly of mycolic acid. In addition, it was confirmed by TEM observation that the strain into which peroxidase was introduced had a relatively larger cell size than the cell size of the wild-type strain (FIG. 3).

In the present invention, the recombinant Rhodococcus opacus may have a reduced cell membrane thickness compared to the parent strain.

In another aspect, the present invention relates to a method of producing free fatty acids by cell membrane lipid peroxidation comprising the steps of:
(a) culturing the recombinant Rhodococcus opacus to produce free fatty acids by cell membrane lipid peroxidation; and
(b) recovering the produced free fatty acid.

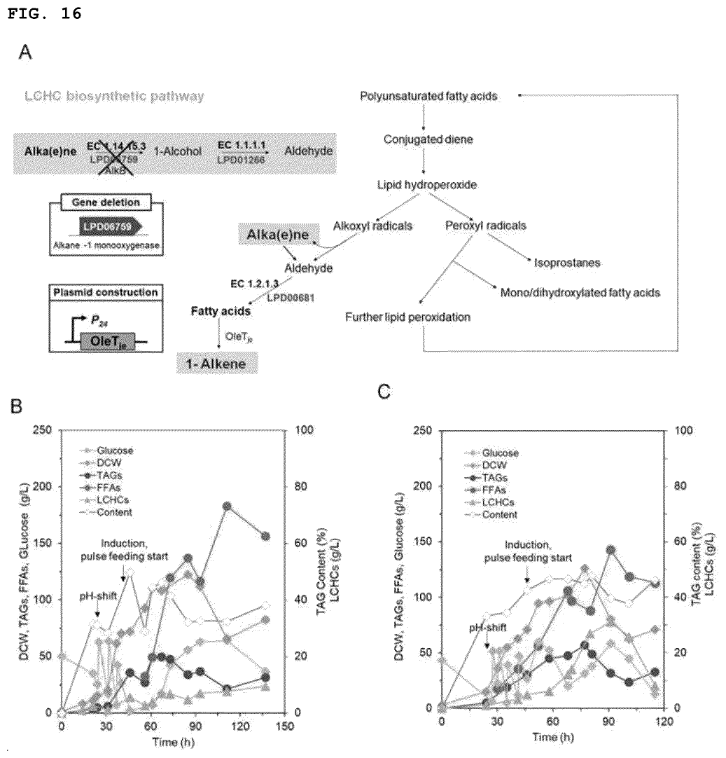

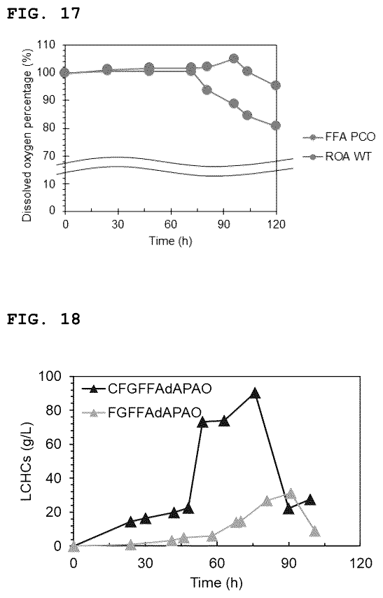

In one example of the present invention, peroxidase was used to induce artificial lipid peroxidation and increase the production of FFAs. Incidentally, it was confirmed that, since peroxidase could catalyze the alkane/alkene biosynthesis reaction, the strain produced hydrocarbons through fed-batch culture thereof (see FIG. 16).

In another aspect, the present invention is directed to a method of producing triacylglycerols by cell membrane lipid peroxidation comprising the steps of:
(a) culturing the recombinant bacterium to triacylglycerols by cell membrane lipid overproduce peroxidation starting from the initial stage of the culturing; and
(b) recovering the produced triacylglycerols.

In one example of the present invention, it was confirmed that peroxidase induced artificial lipid peroxidation to produce free fatty acid (FFA) and, at the same time, accumulate large amounts of triacylglycerols (TAGS), and the accumulated TAGs were additionally converted into FFAs through lipase overexpression.

In another aspect, the present invention is directed to a method for producing hydrocarbons comprising the steps of:
(a) culturing the recombinant Rhodococcus opacus to produce hydrocarbons; and
(b) recovering the produced hydrocarbons.

In the present invention, step (a) may comprise additionally supplementing carbon dioxide.

In one example of the present invention, peroxidase was used to induce artificial lipid peroxidation and increase the production of FFAs. Incidentally, it was confirmed that the oxidation-reduction energy that was increased by artificial lipid peroxidation produced polyolefin through polymerization of alkenes with vinyl (see FIG. 19).

In another aspect, the present invention is directed to a method for producing polyolefin comprising the steps of:
(a) culturing the recombinant Rhodococcus opacus to produce polyolefin; and
(b) recovering the produced polyolefin.

In the present invention, step (a) may comprise additionally supplementing carbon dioxide.

In one example of the present invention, for the production of medium-chain-length polyhydroxyalkanoates, a recombinant Rhodococcus opacus containing the medium-chain-length (MCL) polyhydroxyalkanoate synthase encoded by phaC gene from Pseudomonas and the (R)-specific enoyl-CoA hydratase encoded by phaJ was constructed, and successful production of medium-chain-length polyhydroxyalkanoates by fed-batch culture of the recombinant strain was confirmed (see FIGS. 20 to 24).

In another aspect, the present invention relates to a method for producing polyhydroxyalkanoates comprising the steps of:
(a) culturing the recombinant Rhodococcus opacus to produce polyhydroxyalkanoates; and
(b) recovering the produced polyhydroxyalkanoates.

In one example of the present invention, it was determined that a vinyl polymer could be synthesized through radical polymerization of carbon-carbon bonded alkenes containing monomers produced by artificial lipid peroxidation, and production of vinyl polymers by the recombinant strain FFAdA PCO AFLO OLET was confirmed (see FIG. 19).

In another aspect, the present invention is directed to a method for producing vinyl polymers comprising the steps of:
(a) culturing the recombinant Rhodococcus opacus to produce vinyl polymers; and
(b) recovering the produced vinyl polymers.

### EXAMPLES

Hereinafter, the present invention will be described in more detail with reference to examples. These examples are only for illustrating the present invention, and it will be obvious to those skilled in the art that the scope of the present invention is not to be construed as being limited by these examples.

### Example 1: Construction of Recombinant Microorganism into which Artificial Lipid Peroxidation System has been Introduced

In this example, to construct an artificial lipid peroxidation system, a fungal peroxidase gene (SEQ ID NO: 2) called lignin peroxidase (EC 1.11.1.14) from Phanerochaete carnosa was introduced into the R. opacus PD630 strain (DSMZ, Deutsche Sammlung von Mikroorganismen und Zellkulturen GmbH).

Amino acid sequence of lignin peroxidase (SEQ ID NO: 1)

MAFKQLIVAISIALSLQVTQAVVLKDKRATCSNGASVGDESCCAWFDVLD
DIQQNLFNGGQCGAEAHESIRLVFHDSIAISPAMEAQGKFGGGGADGSII
LFDEIETAFHPNIGLDEVVNLQKPFIAKHGVTPGDFIAFAGAVAMSNCPG
APQMNFFTGRAPATQAAPDGLVPEPFHTVDQIIDRVNDAGQFDELELVWM
LSAHSVAASNDVDPTVQGLPFDSTPGVFDSQFFVETQLRGVLFPGSGGNQ
GEVESGLAGEIRLQSDHTLARDSRTACEWQSFVNNQSKLTSDFQFIFLAL
TQLGQNPDAMTDCSAVIPISKPIPGNGPFSFFPAGKTSADVEQACASTPF
PSLTTLPGPTTSVARIPPPPGA

Nucleotide sequence of lignin peroxidase gene (SEQ ID NO: 2)

atggccttcaagcagctcatcgtcgcaatttccatcgcgctctctctcca
ggtcactcaagctgtcgtgctgaaggacaagcgcgccacctgctcaaacg
gtgccagcgtcggcgacgaatcgtgctgcgcatggttcgacgtcctcgac
gacatccagcagaacctcttcaacggaggccaatgcggcgctgaagccca
cgagtccatccgactcgtcttccacgactccatcgccatctctcctgcaa
tggaggcacaggggaagtttggtggtggaggtgccgacggctccatcatt
ctcttcgatgagattgagaccgcattccacccgaacatcggtctcgacga
agtcgtcaatcttcagaagccattcatcgctaaacacggtgtcacccccg
gcgattttattgccttcgccggtgcagtcgccatgagcaactgccccggt
gccccgcagatgaacttcttcactggtcgcgctcctgctacccaagccgc
gcccgatggtctcgttcccgagccgttccacactgtcgaccagatcattg
accgtgtcaacgatgccggccagttcgatgagctcgagcttgtctggatg
ctctcggcccactctgtcgcggcctccaacgacgtcgacccgactgtcca
aggcctgccgttcgactccactcccggcgtctttgactcccagttcttcg
tcgagacccagctacgcggtgtgctcttccccggctctgggggcaaccaa
ggcgaggtcgagtctggactcgcgggtgaaatccgtcttcagtccgacca
taccctcgcgcgcgactcgcgcacggcctgcgagtggcagtcctttgtca
acaaccagtcgaagctgacgagcgacttccagttcatcttcctcgcgctc
acacagctcggccagaacccggacgcgatgaccgactgctcggccgtcat
tccgatctccaagcccatccccggcaacggcccgttctcgttcttccccg
ccggcaagaccagcgccgacgtcgagcaggcttgcgcgtccacccccttc
ccgagcctcacgactctccctggccccacgacttcggtcgctcgaatccc
accacctccaggtgcttaat

Bacterial peroxidase has high specificity for native lipid peroxidation and thus can cause premature cell death, which is the purpose of existing lipid peroxidation. This enzyme is catalyzed by both phenolic and non-phenolic components after extracellular secretion. In order to confirm the possibility of extracellular secretion of the exogenous peroxidase after introduction into R. opacus, the signal peptide was classified. A recombinant strain obtained by introducing the lignin peroxidase from Phanerochaete carnosa into wild-type R. opacus was named ROA PCO.

The signal peptide of peroxidase in R. opacus was predicted using SignalP 5.0 (Almagro Armenteros et al., Nat Biotechnol 37:420-423, 2019), a deep neural network-based method, and classified as a Sec/SPI secretion signal peptide. Model with a probability of 0.95 (Almagro Armenteros et al. 2019, FIGS. S5 and S6). The Sec/SPI protein type indicates that the protein will be transported to an extracellular location by the Sec translocon and cleaved by type I signal peptidase, a membrane-bound endopeptidase (van Roosmalen et al., Molecular Cell Research, 1694 (1-3): 279-297, 2004). Therefore, peroxidase from P. carnosa was used in R. opacus without signal peptide manipulation.

Accordingly, a system for expressing the lignin peroxidase was constructed by introducing a constitutive promoter.

### Constitutive Promoter and Untranslated Sequence

(SEQ ID NO: 63)
tgtgcgggctctaacacgtcctagtatggtaggatgagcaacatttcga
cgccgagagattcgccgcccgaaatgagcacgatccgcatgcttaatta
agaaggagatatacat

For the promoter sequence and lignin peroxidase sequence, sequence fragments were obtained using gene synthesis, and then the corresponding sequences were combined by Gibson assembly with the platform plasmid (pCH) obtained by PCR amplification using pCH_invR_pco/PCH_invF_pco primers.

TABLE 1

| Name | Nucleotide sequence (5′−>3′) |
|---|---|
| pCH_invR_pco | ATGTATATCTCCTTCTTAATTAAGCATG |
| | (SEQ ID NO: 11) |
| pCH_invF_pco | TTTCACCCTAAATTCGAGAGATT |
| | (SEQ ID NO: 12) |

To confirm the effectiveness of intentionally induced lipid peroxidation for free fatty acid production, fed-batch culture was performed.

### Culture Conditions

Fed-batch culture was performed at 30° C. in a 5-L MARADO-05D-PS fermentor (BioCNS) containing 1.8 L of MC medium. A culture (0.3 L) was prepared by placing 1 mL of a culture (obtained by culturing in 5 mL of medium and passaging the culture 24 to 48 hours after inoculation) in a 250-ml Erlenmeyer flask containing 100 mL of medium and culturing the same for about 24 to 48 hours. The initial OD at 600 nm after inoculation was about 0.5 to 1. The initial pH of the medium was set at 6.4, then adjusted to 7.0 after 24 hours, and kept constant at 7.0 by adding 5M NaOH. Air was continuously added through a 0.2-μm HEPA filter (Millipore) at a rate of 2 L/min. The dissolved oxygen concentration was maintained at 40% air saturation by automatically adjusting the air and pure oxygen flow rates to a constant total gas flow rate of 1 vvm at an initial agitation speed of 300 rpm. The agitation speed was automatically adjusted up to 700 rpm to maintain the dissolved oxygen concentration at 40% air saturation. For fed-batch culture, when the residual glucose concentration in the bioreactor was about 15 g/L, 100-mL feed solution was manually added, and after acetamide was fed, 80 g of glucose and 0.5 g of MgSO₄·7H₂O were contained in the 100-mL feed solution. After acetamide was fed, a strategy of adding glucose in proportion to the glucose consumption rate of each recombinant strain was used. Acetamide was used as an inducer to overexpress intracellular lipase and was added to a final concentration of 0.17 M. Km was added to the recombinant Rhodococcus opacus strain at a concentration of 100 mg/L, and Antifoam 204 (Sigma-Aldrich) was manually added to each vessel to suppress foam formation.

### Culture Medium Components

Medium components per liter: 40 g glucose, 3.3088 g KH₂PO₄, 7.9552 g K₂HPO₄, 14.2 g (NH₄)₂SO₄, 2 g MgSO₄7H₂O, 2.86 mg H₃BO₄, 15 mg CaCl₂, 1 ml stock A solution, and 1 ml of trace metal solution. Stock A solution components per liter: 2 g NaMoO₄2H₂O and 5 g FeNaEDTA. Trace metal solution components per liter: 0.5 g FeSO₄7H₂O, 0.4 g ZnSO₄H₂O, 0.02 g MnSO₄H₂O, 0.01 g NiCl₂6H₂0, 0.05 g CuSO₄5H₂O, 0.01 g MnCl₂, and 0.05 g CoCl₂6H₂O.

### Production Analysis

After culturing, production analysis was conducted under the following conditions. After fed-batch culture, the culture was centrifuged at 4,000 g for 30 minutes, salts in the supernatant were removed, and the supernatant was diluted. At this time, a sample before 60 hours of culture was diluted 20-fold, and a sample after 60 hours of culture was diluted 100-fold. The diluted sample was dried in an oven at 70° C. to obtain a sample for GC. The dried sample was subjected to a methanolysis reaction according to the same method as described in Kurosawa et al., J. Biotechnol. 147, 212-218, 2010.

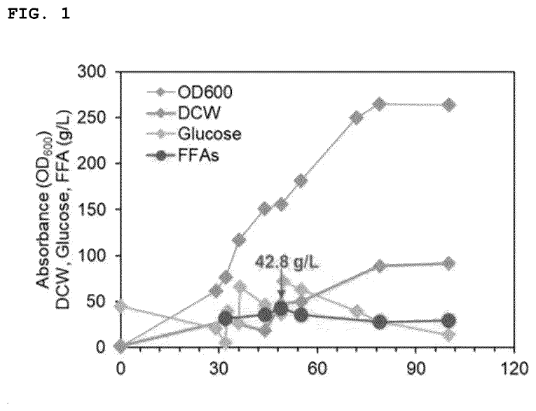

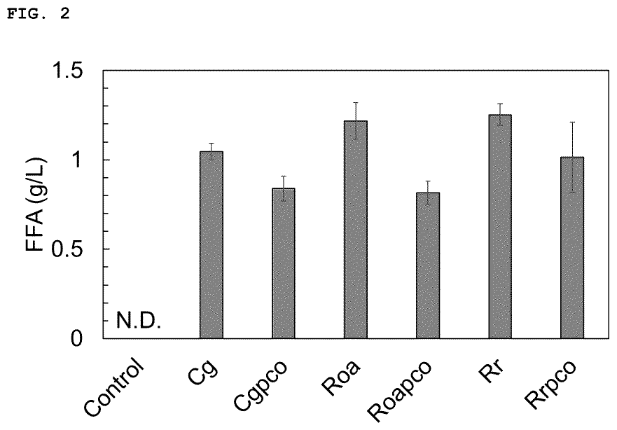

As a result, as shown in FIG. 1, the ROA PCO strain overexpressing the lignin peroxidase from Phanerochaete carnosa produced 42.8 g/L of free fatty acids (FFA) from glucose for 49 hours in fed-batch culture. It was confirmed that the concentration of free fatty acids slightly decreased or was maintained after reaching the highest production. This is because free fatty acids were also utilized as carbon and energy sources in the metabolism of R. opacus. It was confirmed that, through the fed-batch culture, the fatty acid metabolic pathway for biomass production, especially cell membrane production, was successfully changed to an extracellular free fatty acid metabolic pathway. In addition, as shown in FIG. 2, Corynebacterium glutamicum, Rhodococcus opacus, and Rhodococcus ruber, which are representative Gram-positive bacteria, were each grown in LB medium for 48 hours, and then only the cells were collected by centrifugation and added to the fermentation supernatant in which the lignin peroxidase from Phanerochaete carnosa had been overexpressed for 14 hours in fed-batch culture. The cells in the fermentation broth were centrifuged at 4,000 g for 30 minutes, and then only the supernatant was collected to evaluate the cell wall degradation effect of the lignin peroxidase from Phanerochaete carnosa secreted in the fermentation supernatant. Accordingly, it was confirmed that, when the centrifuged Corynebacterium glutamicum, Rhodococcus opacus, and Rhodococcus ruber cells were resuspended in the supernatant and incubated in vitro for 2 hours, free fatty acids were produced.

### Example 2: Confirmation of Changes in Cell Membrane Thickness and Mycomembrane by Artificial Lipid Peroxidation

In order to understand the exact mechanism of glycolysis in the R. opacus into which the artificial lipid peroxidation system constructed in Example 1 has been introduced, the cell membranes before and after lipid peroxidation were compared by TEM observation.

The present inventors examined whether the cell membrane thickness of the ROA PCO strain decreased due to lipid peroxidation, because the peroxidase secreted from the recombinant ROA PCO strain reacted with the cell membrane, centered on the cell membrane outer membrane composed mainly of mycolic acid.

As a result, as shown in FIG. 3, it was confirmed by TEM observation that the strain into which peroxidase had been introduced had a relatively larger cell size than the cell size of the wild-type strain. Additionally, cell membranes and walls of various sizes were observed. In the wild-type strain, the cell membrane and cell wall were relatively thick, and thus relatively white compared to the cytoplasm, whereas in the ROA PCO strain, a significant amount of the cell membrane, mainly the outer membrane, was removed, and thus only a black layer was observed. These morphological changes indicate that lipid peroxidation occurred in the ROA PCO strain overexpressing peroxidase without cell damage and that a redirection of fatty acid metabolism from intracellular fatty acids (FA) to extracellular free fatty acids (FFA) occurred.

In the wild type strain, the average thickness of the cell membrane and wall layer was found to be between 33.45 nm (±9.48 nm, P-value <0.0001) and 18.43 nm (±4.93 nm, P-value <0.0001). The ROA PCO strain was analyzed to be a peroxidase and laccase-like multicopper oxidase-overexpressing strain having an average thickness of cell membrane and wall layer of 12.31 nm (±2.90 nm. P-value <0.0001).

In addition, as a result of observing cells in the exponential phase in the exponential stage by TEM, the process of peroxidase secretion and lipid peroxidation could be seen.

In addition, changes in the components of the mycomembrane layer of the ROA PCO strain were examined, and the results are shown in FIG. 4.

For the TLC analysis in FIG. 4, a silica gel 60 F254 aluminum sheet (Merck, No. 5554) was used. Small sheets (10 cm×3 cm) were prepared by cutting from large sheets (20 cm×20 cm), and one-dimensional TLC was performed using a solution of chloroform:methanol:formic acid (63:30.6:0.4, v/v) as a developing solvent. All samples were prepared dissolved in a chloroform:methanol (2:1) solution. TLC fraction images were visualized using CAMAG TLC Visualizer 2, and target compounds such as fluorescent compounds were visualized by emitting long-wavelength UV light (366 nm).

### Example 3: Production of High Concentrations of Free Fatty Acids Through Metabolic Engineering Study of Artificial Lipid Peroxidation-Based Recombinant R. opacus PD630 and Optimization of Culture Conditions

In order to construct a recombinant strain that produces higher concentrations of free fatty acids, the chromosome of R. opacus was also manipulated in addition to introducing the lignin peroxidase gene.

First, in order to inhibit further reaction of free fatty acid (FFA) with acyl-CoA synthetase encoded by the fadD gene, six fadD genes on the chromosome of R. opacus were deleted.

Deletion of six fadD (LPD00108, LPD00166, LPD00355, LPD04271, LPD05217, and LPD06856) genes was performed in the same manner as described in Kim et al., Nature Chem. Biol. 15, 721-729, 2019.

The left and right DNA regions flanking each fadD gene were individually amplified by PCR using the following primer sets.

TABLE 2

| Name | Nucleotide sequence (5′→3′) |
|---|---|
| fadD00108_LA_F | TACGCCAAGCTTGCATGCCTGCAG |
| | CCGAAAGAGGTCCGCGAGTTCGCA |
| | SEQ ID NO: 13) |
| fadD00108_LA_R | GGGAGACTTTCTCGTGATGG |
| | (SEQ ID NO: 14) |
| fadD00108_RA_F | CCATCACGAGAAAGTCTCCCTACCGAAAGCACAA |
| | GCGTGAGCGAC |
| | (SEQ ID NO: 15) |
| fadD00108_RA_R | ATTCGAGCTCGGTACCCGGGGATCCATCTCGGTG |
| | ATGCGCCGCGACCA |
| | (SEQ ID NO: 16) |
| fadD00166_LA_F | TACGCCAAGCTTGCATGCCTGCAG |
| | CAGGTGCCGGOTGGTCAATCGTCC |
| | (SEQ ID NO: 17) |
| fadD00166_LA_R | GCAGTCTCCAAGATCGACGTGTTG |
| | (SEQ ID NO: 18) |
| fadD00166_RA_F | ACGTCGATCTTGGAGACTGCAAGCGGAGCCTCCG |
| | ATGACCTT |
| | (SEQ ID NO: 19) |
| fadD00166_RA_R | ATTCGAGCTCGGTACCCGGGGATCCAGTGCGTCG |
| | TAGGTCATCGCCC |
| | (SEQ ID NO: 20) |
| fadD00355_LA_F | TACGCCAAGCTTGCATGCCTGCAGACACCGGCAA |
| | CATGGAATTGCTGC |
| | (SEQ ID NO: 21) |
| fadD00355_LA_R | CCGCGGACTTCTTTCAGTACCGGGG |
| | (SEQ ID NO: 22) |
| fadD00355_RA_F | GTACTGAAAGAAGTCCGCGGAGACCACCATGCCC |
| | ACCGATTCCG |
| | (SEQ ID NO: 23) |
| fadD00355_RA_R | ATTCGAGCTCGGTACCCGGGGATCCGATTCCGAA |
| | GATCAGCTTCCGG |
| | (SEQ ID NO: 24) |
| fadD04271_LA_F | TACGCCAAGCTTGCATGCCTGCAGTCACTCCACG |
| | CTCACCCAGGCCAC |
| | (SEQ ID NO: 25) |
| fadD04271_LA_R | CCTCTATGTCGGGTGCAGTGCGGC |
| | (SEQ ID NO: 26) |
| fadD04271_RA_F | CACTGCACCCGACATAGAGGGGAGCCGGACATGA |
| | CCGACAGTTAT |
| | (SEQ ID NO: 27) |
| fadD04271_RA_R | ATTCGAGCTCGGTACCCGGGGATCCTCATGTAAT |
| | GCAGGTCGCTGTGG |
| | (SEQ ID NO: 28) |
| fadD05217_LA_F | TACGCCAAGCTTGCATGCCTGCAGCTCCAGAACG |
| | TACGAACCGAAC |
| | (SEQ ID NO: 29) |
| fadD05217_LA_R | GAAAACCTCCGACACTGAAGATA |
| | (SEQ ID NO: 30) |
| fadD05217_RA_F | CTTCAGTGTCGGAGGTTTTCGGCTCGTGAGTACT |
| | TATCAAC |
| | (SEQ ID NO: 31) |
| fadD05217_RA_R | ATTCGAGCTCGGTACCCGGGGATCCACCTTGATG |
| | GTGCCCACACCGT |
| | (SEQ ID NO: 32) |
| fadD06856_LA_F | TACGCCAAGCTTGCATGCCTGCAGTGAACGCCTG |
| | AGTCCGACAGCACG |
| | (SEQ ID NO: 33) |
| fadD06856_LA_R | GATGAGTCCTCCGTCCCGGTGCCGC |
| | (SEQ ID NO: 34) |
| fadD06856_RA_F | ACCGGGACGGAGGACTCATCCCGTCATGACCATC |
| | GAATTCGAGTC |
| | (SEQ ID NO: 35) |
| fadD06856_RA_F | ATTCGAGCTCGGTACCCGGGGATCCCGAGGAGGT |
| | TGGCCACCGGCACGCG |
| | (SEQ ID NO: 36) |

Second, the native promoters of the LPD01036 and LPD02672 genes encoding MAG lipase were replaced with the inducible promoter Pace, allowing appropriately accumulated triacylglycerols (TAG) to be hydrolyzed into FFAs. In particular, it is important to optimize lipase overexpression time, because overexpression of lipase has a negative impact on cell viability, while premature TAG conversion to FFAs can lead to overproduction of FFAs, thereby increasing cost competitiveness.

The left and right DNA regions for replacing the native promoters of the LPD01036 and LPD02672 genes with the inducible promoter Pace were individually amplified by PCR with the following primer sets. The corresponding promoter replacement was performed in the same manner as described in (Kim et al., Nature Chem. Biol. 15, 721-729, 2019).

TABLE 3

| Name | Nucleotide sequence (5−>3′) |
|---|---|
| Pace01036_LA_F | TACGCCAAGCTTGCATGCCTGCAGTCGCCACCGTG |
| | AAGTCGACGAGGTT |
| | (SEQ ID NO: 37) |
| Pace01036_LA_R | TTATTTCTGCTAGAAAGCTTGTCAGGCCTCCGGAC |
| | TTCCCGGATC |
| | (SEQ ID NO: 38) |
| Pace01036_RA_F | ATAAGAGAAAGGGAGTCCACATGCCCTTCTTCGAC |
| | GGTGTCAGGA |
| | (SEQ ID NO: 39) |
| Pace01036_RA_R | ATTCGAGCTCGGTACCCGGGGATCCGCCGGTGAAA |
| | GTCAGGTTGACGGGA |
| | (SEQ ID NO: 40) |
| Pace02672_LA_F | TACGCCAAGCTTGCATGCCTGCAGGGGCAGTCCGC |
| | CGAGCGCCTGCGG |
| | (SEQ ID NO: 41) |
| Pace02672_LA_R | TTATTTCTGCTAGAAAGCTTTCTGGCTCTGTCGCA |
| | CCCCGATGGC |
| | (SEQ ID NO: 42) |
| Pace02672_RA_F | ATAAGAGAAAGGGAGTCCACATGGCAGCCATGCCC |
| | TTCTTCGACGG |
| | (SEQ ID NO: 43) |
| Pace02672_RA_R | ATTCGAGCTCGGTACCCGGGGATCCCGGTGACGGC |
| | AAGCCGTTCATCGG |
| | (SEQ ID NO: 44) |

Third, two genes related to NADPH that use the metabolic pathway were deleted. NADPH is a key component of FA extension and glycolytic metabolism for reactive oxygen species (ROS) generation. Since engineering to increase NADPH availability is important to promote FFA overproduction due to lipid peroxidation, two additional genes were deleted: LPD12046 encoding 4-dichlorophenol 6-monooxygenase, and LPD16168 encoding putative 2-dehydropantoate 2-reductase.

The left and right DNA regions flanking the LPD12046 and LPD16168 genes were individually amplified by PCR using the following primer sets.

TABLE 4

| Name | Nucleotide sequence (5−>3′) |
|---|---|
| LPD12046_LA_F | TACGCCAAGCTTGCATGCCTGCAG |
| | ATGCGGAACGGGACCATGG |
| | (SEQ ID NO: 45) |
| LPD12046_LA_R | TTATTTCTGCTAGAAAGCTT |
| | TGCACCTTCTCAAACTCTCTGTGGAC |
| | (SEQ ID NO: 46) |
| LPD12046_RA_F | ATAAGAGAAAGGGAGTCCACATG |
| | TGTCCCGTCCATACACCAGC |
| | (SEQ ID NO: 47) |
| LPD12046_RA_R | ATTCGAGCTCGGTACCCGGGGATCC |
| | GACCCGTTCCCAGCTCGG |
| | (SEQ ID NO: 48) |
| LPD16168_LA_F | TACGCCAAGCTTGCATGCCTGCAGCTGGTCCATCG |
| | TCTGGGAGG |
| | (SEQ ID NO: 49) |
| LPD16168_LA_R | TTATTTCTGCTAGAAAGCTT |
| | CGCGAGGATCACCACGTCA |
| | (SEQ ID NO: 50) |
| LPD16168_RA_F | ATAAGAGAAAGGGAGTCCACATGTGCATGCGTGGT |
| | CATTCTGT |
| | (SEQ ID NO: 51) |
| LPD16168_RA_R | ATTCGAGCTCGGTACCCGGGGATCC |
| | AGGAAACCGACGCCACCC |
| | (SEQ ID NO: 52) |

Finally, to confirm the genotype, next-generation sequencing (NGS) was performed on the strain with the gene deleted. Based on the genomic data, it was confirmed that other redundant sequences were removed due to the high GC content of R. opacus. The engineered strain was named FFA PCO.

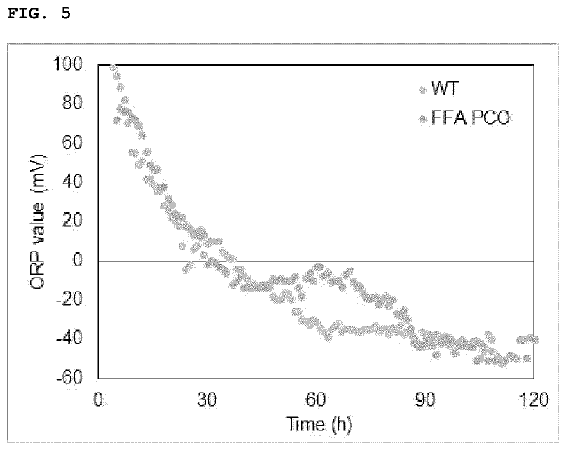

In the present invention, as a result of measuring the ORP value during fed-batch fermentation of the recombinant FFA PCO strain, it could be confirmed that the oxidative stress of the culture was higher than that of the wild-type strain (FIG. 5).

In the present invention, the ORP value denotes oxidation-reduction potential, and the unit of measurement using the ORP probe is millivolt (mV). Oxidizing agents with oxidizing power increase the ORP measurement value, and representative examples thereof include hydrogen peroxide, ozone, and light. However, in the present invention, oxidizing agents refer to oxidizing agents generated by the recombinant Rhodococcus opacus within cells. Reducing agents with reducing power reduces the ORP measurement value, and as microorganisms grow, the reducing power increases and the ORP measurement value tends to decrease.

In FIG. 5, it can be seen that the ORP measurement value of the recombinant FFA PCO strain increased compared to the ORP measurement value of wild-type Rhodococcus opacus at 45 to 90 hours of culture at which the artificial lipidation system of the cell was actively expressed in the cell, suggesting that the oxidative power of the cells was increased by the artificial lipidation system. This indicates that the artificial lipid peroxidation system was successfully introduced.

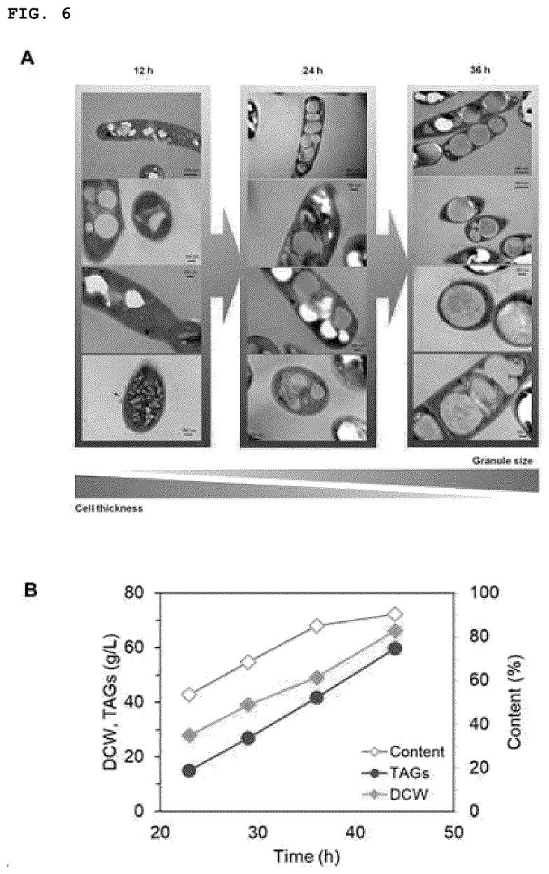

To confirm the FFA overproduction of the FFA PCO strain, fed-batch culture was performed in the same manner as Example 1, and the results of the initial fed-batch culture (20 to 44 hours) are shown in FIG. 6.

The recombinant FFA PCO strain produced 113.92 g/L of FFAs from glucose for 75 hours without overexpressing lipase, and the production of FFAs was significantly increased while the production of triacylglycerols (TAGs) decreased, suggesting that TAGs released from cells due to its high TAG content were directly converted into extracellular FFAs by the synergistic effects of physical degradation (by agitation) and chemical degradation. That is, additional in vitro hydrolysis is induced by the reaction of extracellular lipase and TAGs, and TAGs are secreted due to high TAG content and cell lysis. Moreover, the excretion of TAGs may occur due to enhanced cell membrane permeability induced by artificial lipid peroxidation.

### Example 4: Additional Introduction of Laccase-Like Multicopper Oxidase (LMCO) Enzyme Gene for Cell Viability and Activity of Recombinant Strain

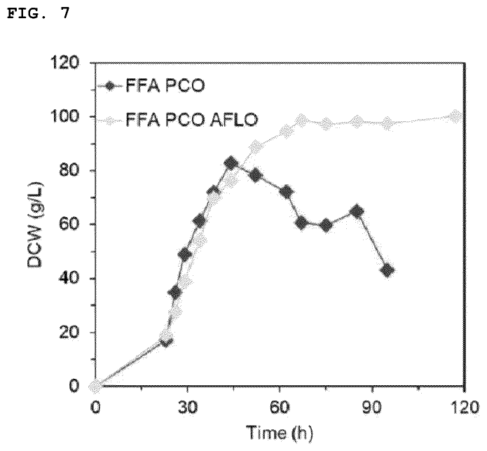

The viability of the recombinant peroxidase-overexpressing strain was poor compared to the wild-type strain. During fed-batch culture, the highest dry cell weight (DCW) was achieved at 44 hours, reaching 82.7 g/L DCW with 40.99 g/L TAG (FIG. 6B). After 44 hours, DCW started to decrease and reached 43.1 g/L within 95 hours with 18.04 g/L TAG (FIG. 7).

Assuming that this early decrease in cell activity was attributed to an unstable and unbalanced radical system lethal to cell, the present inventors designed a system that introduces a new enzyme to further stabilize the cell state.

To overcome the early decrease in biomass during fermentation and to promote the cell membrane decomposition reaction by supplying hydrogen peroxide (H₂O₂) for balanced radical system, the laccase-like multicopper oxidase (LMCO) gene was additionally introduced into the FFA PCO strain.

In this example, the step to generate H₂O₂ before artificial lipid peroxidation was designed to enhance the electron acceptors of artificial lipid peroxidation. LMCO (EC 1.10.3.2) from Aspergillus flavus, which can be used as a biocatalyst for the assimilation of very long-chain hydrocarbons and polyethylene, was introduced to produce and excrete free fatty acids more effectively.

Accordingly, a newly designed plasmid, PCH PCO AFLO plasmid, was constructed by introducing the laccase-like multicopper oxidase (LMCO) from Aspergillus flavus into the PCH PCO plasmid.

The laccase-like multicopper oxidase (LMCO) sequence fragment from Aspergillus flavus was obtained through gene synthesis, and the sequence fragment synthesized in the order of RBS site-LMCO was combined by Gibson assembly with the platform plasmid fragment obtained by PCR amplification of the previously constructed PCH PCO plasmid using pCH pco_invR_lmco/PCH_pco_invF_lmco primers.

Amino acid sequence of LMCO from Aspergillus flavus (SEQ ID NO: 3)

MAPLKTLVALLSANVLTSVLAELVKFEVDLTWAKGSPDGNLRDMIFVNDQ
FPAPQLTLNQYDDVEFTVNNHMPFNATVHFHGIVQLNTPWSDGVPGLTQK
PILPGGTFTYRWTATEYGTYWYHAHARSLMADGLYGAIWIKCVPRWPSIL
CKD

Nucleotide sequence of LMCO gene from Aspergillus flavus (SEQ ID NO: 4)

atggcgccgctgaaaaccctggtggcgctgctgagcgcgaacgtgctgac
cagcgtgctggcggaactggtgaaatttgaagtggatctgacctgggcga
aaggcagcccggatggcaacctgcgcgatatgatttttgtgaacgatcag
tttccggcgccgcagctgaccctgaaccagtatgatgatgtggaatttac
cgtgaacaaccatatgccgtttaacgcgaccgtgcattttcatggcattg
tgcagctgaacaccccgtggagcgatggcgtgccgggcctgacccagaaa
ccgattctgccgggcggcacctttacctatcgctggaccgcgaccgaata
tggcacctattggtatcatgcgcatgcgcgcagcctgatggcggatggcc
tgtatggcgcgatttggattaaatgcgtgccgcgctggccgagcattctg
tgcaaagat

### Synthesized RBS Sequence

(SEQ ID NO: 53)
ctcgcagccggtggaaaggaggtctat

TABLE 5

| Name | Nucleotide sequence (5′−>3′) |
|---|---|
| pCH pco_invR_lmco | ATAGACCTCCTTTCCACCGGCTGCGAGATTAA |
| | GCACCTGGAGGTGGT |
| | (SEQ ID NO: 54) |
| pCH pco_invF_lmco | TTCTTCGCAAAAATCGTCCCC |
| | (SEQ ID NO: 55) |

The recombinant strain obtained by introducing the LMCO from Aspergillus flavus into the above recombinant FFA PCO strain was named FFA PCO AFLO strain.

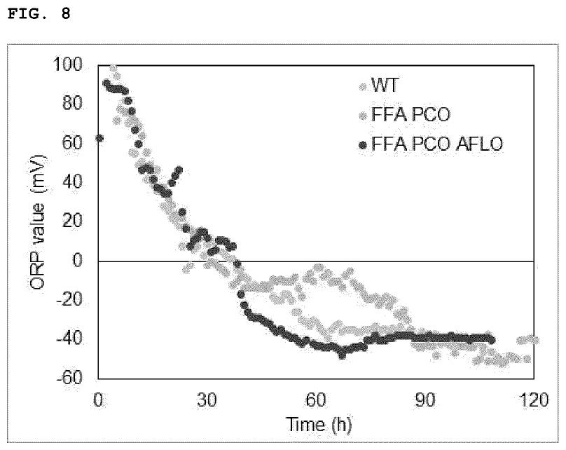

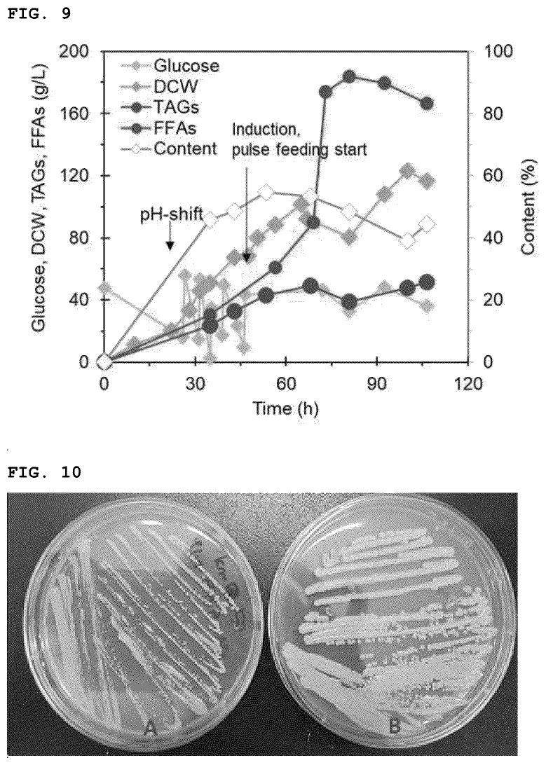

As a result of fed-batch culture of the recombinant FFA PCO AFLO strain in the same manner, as in Example 1, as shown in FIG. 6, it could be confirmed that biomass was maintained even after 44 hours of culture. In addition, through comparison of the ORP values measured during fed-batch culture, it could be confirmed that the oxidation-reduction balance of the system was recovered similarly to the ORP measurement value of the wild-type strain (FIG. 8).

### Example 5: Production of Ultra-High Concentration of Free Fatty Acids Through Glucose-Adaptive Evolution of Recombinant R. opacus PD630 Based on Artificial Lipid Peroxidation

Free fatty acids, designated as the target product as precursors for biofuels and biopolymers in the present invention, are carbon and energy sources that compete with glucose in cellular metabolism. Due to intrinsic fatty acid metabolism, cellular uptake and oxidation rates of fatty acids may be accelerated, thereby inhibiting additional extracellular free fatty acid accumulation. Accordingly, it is important to maintain the extracellular free fatty acid concentration at a high level by upregulating cellular glucose assimilating metabolism and relatively downregulating fatty acid assimilating metabolism. Therefore, an experiment on the glucose adaptive evolution of cells was conducted to increase the glucose uptake rate compared to the free fatty acid uptake rate.

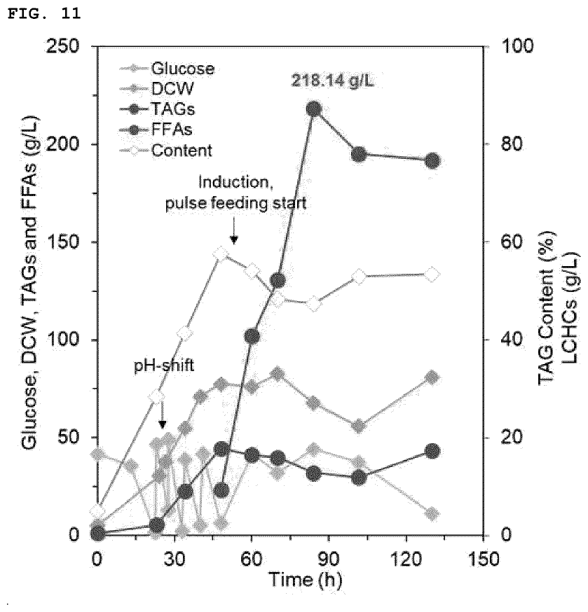

FFA PCO and FFA PCO AFLO strains were grown repeatedly on solid LB plates containing glucose. After confirming the increased change in colony size, each large colony was isolated and seeded into an LB liquid medium. As a result, as shown in FIGS. 10 and 11, a strain showing the ability to produce ultra-high concentration free fatty acids (218.14 g/L) was identified as a result of fed-batch culture, and this strain was named FG FFA PCO.

### Example 6: Biofuel and Hydrocarbon Overproduction Through Additional Metabolic Engineering Study on Recombinant R. opacus PD630 Based on Artificial Lipid Peroxidation

Monooxygenase, peroxidase, and peroxygenase are enzymes that can generate long-chain hydrocarbons by inserting a reactive oxidation source into free fatty acids. In the present invention, peroxidase was used to induce artificial lipid peroxidation and increase the production of FFA. Incidentally, it was confirmed that, since the peroxidase could catalyze the alkane/alkene biosynthesis reaction, the strain was able to produce hydrocarbons during the fed-batch culture thereof (FIG. 12).

### Culture Conditions

Fed-batch culture was performed at 30° C. in a 5-L MARADO-05D-PS fermentor (BioCNS) containing 1.8 L of MC medium. A seed culture (0.3 L) was prepared by placing 1 mL of a seed culture (obtained by culturing in 5 mL of LB medium and incubating the culture 24 to 48 hours after inoculation) in a 250-ml Erlenmeyer flask containing 100 mL of LB medium and culturing the same for about 24 to 48 hours. The initial OD at 600 nm after inoculation was about 0.5 to 1. The initial pH of the medium was set at 6.4, then adjusted to 7.0 after 24 hours, and kept constant at 7.0 by adding 5M NaOH. Air was continuously added through a 0.2-μm HEPA filter (Millipore) at a rate of 2 L/min. The dissolved oxygen concentration was maintained at 40% air saturation by automatically adjusting the air and pure oxygen flow rates to a constant total gas flow rate of 1 vvm at an initial agitation speed of 300 rpm. The agitation speed was automatically adjusted up to 700 rpm to maintain the dissolved oxygen concentration at 40% air saturation. For fed-batch culture, when the residual glucose concentration in the bioreactor was about 15 g/L, 100-mL feed solution including 80 g of glucose and 1 g of MgSO₄·7H₂O was manually added. After acetamide was supplemented for induction, a strategy of adding glucose in proportion to the glucose consumption rate of each recombinant strain was used. Acetamide was used as an inducer to overexpress intracellular lipase and was added to a final concentration of 0.17 M. As antibiotics, kanamycin was added to the recombinant R opacus strain at a concentration of 50 mg/L, and Antifoam 204 (Sigma-Aldrich) was manually added to each vessel to suppress foam formation.

### Culture Medium Components

Medium components per liter: 40 g glucose, 3.3088 g KH₂PO₄, 7.9552 g K₂HPO₄, 14.2 g (NH₄)₂SO₄, 2 g MgSO₄7H₂O, 2.86 mg H₃BO₄, 15 mg CaCl₂, 1 ml stock A solution, and 1 ml of trace metal solution. Stock A solution components per liter: 2 g NaMoO₄2H₂0 and 5 g FeNaEDTA. Trace metal solution components per liter: 0.5 g FeSO₄7H₂O, 0.4 g ZnSO₄H₂O, 0.02 g MnSO₄H₂O, 0.01 g NiCl₂6H₂0, 0.05 g CuSO₄5H₂O, 0.01 g MnCl₂, and 0.05 g CoCl₂6H₂O.

As a result, as shown in FIG. 14A, the FG FFA PCO strain, which was subjected to glucose adaptive evolution after the peroxidase gene from P. carnosa was introduced thereinto, showed a hydrocarbon titer of 3.34 g/L at 70 hours using glucose as a carbon source. Additionally, as shown in FIG. 13B, the FG FFA PCO AFLO strain produced 5.18 g/L of hydrocarbons at 46 hours. This is because the strain was able to maintain its high cell density through stabilization of oxidation-reduction potential by introducing laccase-like multicopper oxidase (LMCO), and thus had longer cell viability.

In this example, in order for the recombinant strain to produce a further increased amount of hydrocarbons, a gene (SEQ ID NO: 6) encoding the OleT enzyme, which catalyzes the removal of carbon dioxide from variable chain-length fatty acids to form 1-alkenes, was introduced into the FFA PCO AFLO strain (FIGS. 13B, 16A and 16B).

Accordingly, a system for expressing the oleT gene was constructed by introducing a constitutive promoter.

### Constitutive Promoter and Untranslated Sequence

(SEQ ID NO: 56)
tgtgcgggctctaacacgtcctagtatggtaggatgagcaacatttcgac
gccgagagattcgccgcccgaaatgagcacgatccgcatgcttaattaag
aaggagatatacat

For the promoter sequence and oleT gene sequence, sequence fragments were obtained using gene synthesis, and then the corresponding sequences were combined by Gibson assembly with the platform plasmid (pNVs) obtained by PCR amplification using pNVs_invR_Ole/PNVs_invF_Ole primers.

TABLE 6

| Name | Nucleotide sequence (5′−>3′) |
|---|---|
| pNVs_invR_Ole | CTGAATGGCGGGAGTATGAAAA |
| | (SEQ ID NO: 57) |
| pNVs_invF_Ole | TACAACGTCGTGACTGGGAAA |
| | (SEQ ID NO: 58) |

Amino acid sequence of OleT enzyme (SEQ ID NO: 5)

MATLKRDKGLDNTLKVLKQGYLYTTNQRNRLNTSVFQTKALGGKPFVVVT
GKEGAEMFYNNDVVQREGMLPKRIVNTLFGKGAIHTVDGKKHVDRKALFM
SLMTEGNLNYVRELTRTLWHANTQRMESMDEVNIYRESIVLLTKVGTRWA
GVQAPPEDIERIATDMDIMIDSFRALGGAFKGYKASKEARRRVEDWLEEQ
IIETRKGNIHPPEGTALYEFAHWEDYLGNPMDSRTCAIDLMNTFRPLIAI
NRFVSFGLHAMNENPITREKIKSEPDYAYKFAQEVRRYYPFVPFLPGKAK
VDIDFQGVTIPAGVGLALDVYGTTHDESLWDDPNEFRPERFETWDGSPFD
LIPQGGGDYWTNHRCAGEWITVIIMEETMKYFAEKITYDVPEQDLEVDLN
SIPGYVKSGFVIKNVREVVDRT

Nucleotide sequence of oleT gene (SEQ ID NO: 6)

atggcaacacttaagagggataagggcttagataatactttgaaagtatt
aaagcaaggttatctttacacaacaaatcagagaaatcgtctaaacacat
cagttttccaaactaaagcactcggtggtaaaccattcgtagttgtgact
ggtaaggaaggcgctgaaatgttctacaacaatgatgttgttcaacgtga
aggcatgttaccaaaacgtatcgttaatacgctttttggtaaaggtgcaa
tccatacggtagatggtaaaaaacacgtagacagaaaagcattgttcatg
agcttgatgactgaaggtaacttgaattatgtacgagaattaacgcgtac
attatggcatgcgaacacacaacgtatggaaagtatggatgaggtaaata
tttaccgtgaatctatcgtactacttacaaaagtaggaacacgttgggca
ggcgttcaagcaccacctgaagatatcgaaagaatcgcaacagacatgga
catcatgatcgattcatttagagcacttggtggtgcctttaaaggttaca
aggcatcaaaagaagcacgtcgtcgtgttgaagattggttagaagaacaa
attattgagactcgtaaagggaatattcatccaccagaaggtacagcact
ttacgaatttgcacattgggaagactacttaggtaacccaatggactcaa
gaacttgtgcgattgacttaatgaacacattccgcccattaatcgcaatc
aacagattcgtttcattcggtttacacgcgatgaacgaaaacccaatcac
acgtgaaaaaattaaatcagaacctgactatgcatataaattcgctcaag
aagttcgtcgttactatccattcgttccattccttccaggtaaagcgaaa
gtagacatcgacttccaaggcgttacaattcctgcaggtgtaggtcttgc
attagatgtttatggtacaacgcatgatgaatcactttgggacgatccaa
atgaattccgcccagaaagattcgaaacttgggacggatcaccatttgac
cttattccacaaggtggtggagattactggacaaatcaccgttgtgcagg
tgaatggatcacagtaatcatcatggaagaaacaatgaaatactttgcag
aaaaaataacttatgatgttccagaacaagatttagaagtggacttaaac
agtatcccaggatacgttaagagtggctttgtaatcaaaaatgttcgcga
agttgtagacagaacataa

An additional glucose adaptive evolution process was performed on the recombinant strain into which the gene encoding the OleT from Jeotgalicoccus sp. had been introduced. The resulting strain was named FG FFA PCO AFLO OLET.

As a result, as shown in FIG. 13B, it was confirmed that the FG FFA PCO AFLO OLET strain converted free fatty acids into alkanes and alkenes, and resulted in producing 5.59 g/L of hydrocarbons for 46 hours and 9.56 g/L of hydrocarbons for 137 hours from glucose by fed-batch culture.

In addition, since the inherent long-chain hydrocarbon degradation pathway in Rhodococcus can interfere with the intracellular accumulation of hydrocarbons, the present inventors constructed the recombinant strain FG FFAdA PCO AFLO OLET wherein the alkB gene (EC 1. 14. 15. 3), a gene encoding an enzyme that degrades hydrocarbons, is deleted. Furthermore, an additional glucose adaptive evolution process was performed on the strain, and the resulting strain was named FG FFAdA PCO AFLO OLET. The left and right DNA regions for deleting the alkB gene in the recombinant strain FG FFA PCO AFLO OLET were individually amplified by PCR using the following primer sets.

TABLE 7

| Name | Nucleotide sequence (5′−>3′) |
|---|---|
| AlkB_LA_F | TACGCCAAGCTTGCATGCCTGCAG |
| | CTGTGGATCTGGTGCACCTC |
| | (SEQ ID NO: 59) |
| AlkB_LA_R | TTATTTCTGCTAGAAAGCTTGATCCGAACCTCCTCGTCC |
| | (SEQ ID NO: 60) |
| AlkB_RA_F | ATAAGAGAAAGGGAGTCCACATG |
| | TGGCCGCCTACCAGTGCC |
| | (SEQ ID NO: 61) |
| AlkB_RA_R | ATTCGAGCTCGGTACCCGGGGATCC |
| | ATAGGGCGGTGCGGGTTCG |
| | (SEQ ID NO: 62) |

As a result, as shown in FIG. 16C, the FG FFAdA PCO AFLO OLET strain produced 31 g/L of hydrocarbons for 91 hours during fed-batch culture. This indicates a glucose yield of 0.1 g/g and a productivity of 0.34 g/L/h at 91 hours of fed-batch culture.

### Example 7: Overproduction of Biofuels, Long Chain Hydrocarbons Through Carbon Dioxide Fixation in Recombinant R. opacus PD630 Based on Artificial Lipid Peroxidation

Since the recombinant strain, artificially inducing lipid membrane peroxidation, developed in the present invention has a higher reduction potential than wild-type R. opacus PD630 during fed-batch culture, it is advantageous for carbon dioxide fixation in the cellular metabolic pathway (see FIGS. 5 and 8). Therefore, carbon dioxide and formic acid as sole carbon and energy sources were supplied to the cells, and carbon dioxide fixation in the cells was confirmed (FIG. 17).

In addition, only carbon dioxide, not formic acid was separately added to the 5 L-scale fed-batch culture with another carbon source, glucose and it showed the improved biomass production versus glucose consumption (FIG. 14) and the increased hydrocarbon production was also confirmed (FIGS. 15 and 18).

### Culture Conditions

Fed-batch culture using carbon dioxide and formic acid as sole carbon and energy sources was performed 32° C. in a 1.3-L Bioflow 110 bioreactor (New Brunswick Scientific) containing 0.5 L of MC medium. A seed culture (75 mL) was prepared by placing 1 mL of a seed culture (obtained by culturing in 5 mL of LB medium and incubating the seed culture 24 to 48 hours after inoculation) in a 250-ml Erlenmeyer flask containing 100 mL of LB medium and culturing the same for about 24 to 48 hours. The initial OD at 600 nm after inoculation was about 0.5 to 1. The initial pH of the medium was set at 6.4, then adjusted to 7.0 after 24 hours, and kept constant at 7.0 by adding 5M NaOH. Air and carbon dioxide were continuously added through a 0.2-μm HEPA filter (Millipore) at rates of 0.5 L/min and 0.05 L/min, respectively. Separate carbon sources other than carbon dioxide and formic acid were not added, and cell metabolism was examined through the dissolved oxygen level in the fermentor (FIG. 17). Antibiotics were selectively supplemented at the following concentrations when necessary: 50 mg/L kanamycin; 100 mg/L streptomycin.

Fed-batch culture using additional carbon dioxide supplementation was performed at 32° C. in a 5-L MARADO-05D-PS fermentor (BioCNS) containing 1.8-L MC medium. A seed culture (0.3 L) was prepared by placing 1 mL of a seed culture (obtained by culturing in 5 mL of LB medium and incubating the culture 24 to 48 hours after inoculation) in a 250-ml Erlenmeyer flask containing 100 mL of LB medium and culturing the same for about 24 to 48 hours. The initial OD at 600 nm after inoculation was about 0.5 to 1. The initial pH of the medium was set at 6.4, then adjusted to 7.0 after 24 hours, and kept constant at 7.0 by adding 5M NaOH. Air and carbon dioxide were continuously added through a 0.2-μm HEPA filter (Millipore) at rates of 2 L/min and 0.2 L/min, respectively. The dissolved oxygen concentration was maintained at 40% air saturation by automatically adjusting the air and pure oxygen flow rates to a constant total gas flow rate of 1 vvm at an initial agitation speed of 300 rpm. The agitation speed was automatically adjusted up to 700 rpm to maintain the dissolved oxygen concentration at 40% air saturation. For fed-batch culture, when the residual glucose concentration in the bioreactor was about 15 g/L, 100-mL feed solution including 80 g of glucose and 1 g of MgSO₄·7H₂O was manually added. After acetamide was supplemented at a final concentration of 0.17 M, a strategy of adding glucose in proportion to the glucose consumption rate of each recombinant strain was used. Acetamide was used as an inducer to overexpress intracellular lipase. Antibiotics were selectively supplemented at the following concentrations when necessary: 50 mg/L kanamycin; 100 mg/L streptomycin. Antifoam 204 (Sigma-Aldrich) was manually added to each vessel to suppress foam formation.

### Culture Medium Components

Medium component per liter: 40 g glucose, 3.3088 g KH₂PO₄, 7.9552 g K₂HPO₄, 14.2 g (NH₄)₂SO₄, 2 g MgSO₄7H₂O, 2.86 mg H₃BO₄, 15 mg CaCl₂, 1 ml stock A solution and 1 ml of trace metal solution. Stock A solution components per liter: 2 g _{NaMoO42H20} and 5 g FeNaEDTA. Trace metal solution components per liter: 0.5 g FeSO₄7H₂O, 0.4 g ZnSO₄H₂O, 0.02 g MnSO₄H₂O, 0.01 g NiCl₂6H₂0, 0.05 g CuSO₄5H₂O, 0.01 g MnCl₂, and 0.05 g CoCl₂6H₂O.

As a result, as shown in FIGS. 15 and 18, it was confirmed that the FG FFA PCO strain and the FG FFAdA PCO AFLO OLET strain were able to produce increased amounts of hydrocarbons through fed-batch culture by additionally supplementing carbon dioxide.

### Example 8: Overproduction of Biopolymers, Polyolefins, and Polyhydroxyalkanoates Through Additional Metabolic Engineering Study on Recombinant R. opacus PD630 Based on Artificial Lipid Peroxidation

The production of polyolefin in the culture medium was examined through analysis of the culture medium of the fed-batch culture of the ultra-high-concentration free fatty acid- and alkene-producing strain developed in the present invention. This is not only the first report of achieving intracellular polymerization of olefins using the recombinant strain derived from R. opacus PD630, but also the first report of producing microorganism-derived polyolefin from glucose through a one-step fermentation process without the additional supplementation of monomers of polyolefins (see FIGS. 19 and 24).

By employing the ultra-high-concentration free fatty acid-producing strain developed in the present invention and establishing a platform for producing diverse biopolymers, the production of medium-chain-length polyhydroxyalkanoates was also performed. This is the first report of producing medium-chain-length polyhydroxyalkanoate using the recombinant strain derived from R. opacus PD630.

Polyolefin and medium-length polyhydroxyalkanoate in the culture were recovered using solvent extraction and precipitation methods. To recover a sustainable biopolymer, the culture was extracted with acetone, a non-halogen solvent, for 36 hours using a Soxhlet apparatus at 50° C., and then the extract concentrate was treated with methanol, thereby recovering the biopolymer from the culture.

For the production of medium-chain-length polyhydroxyalkanoate, the present inventors constructed recombinant Rhodococcus opacus containing the medium-chain-length (MCL) polyhydroxyalkanoate synthase encoded by the phaC gene from Pseudomonas and the (R)-specific enoyl-CoA hydratase encoded by phaJ gene. Additional recombination was performed using the strain constructed in Examples 1 to 6.

Amino acid sequence of PhaC enzyme (SEQ ID NO: 7)

MTDKPAKGSTTLPATRMNVQNAILGLRGRDLLSTLRNVGRHGLRHPLHTA
HHLLALGGQLGRVMLGDTPYQPNPRDARFSDPTWSQNPFYRRGLQAYLAW
QKQTRQWIDESHLNDDDRARAHFLQNLINDALAPSNSLLNPLAVKELFNT
GGQSLVRGVAHLLDDLRHNDGLPRQVDERAFEVGVNLAATPGAVVQRNEL
LELIQYSPMSEKQHARPLLVVPPQINKFYIFDLSATNSFVQYMLKSGLQV
FMVSWRNPDPRHREWGLSSYVQALEEALNACRSISGNRDPNLMGACAGGL
TMAALQGHLQAKKQLRRVRSATYLVSLLDSKFESPASLFADEQTIEAAKR
RSYQRGVLDGGEVARIFAWMRPNDLIWNYWVNNYLLGKTPPAFDILYWNA
DSTRLPAALHGDLLEFFKLNPLTYASGLEVCGTPIDLQQVNIDSFTVAGS
NDHITPWDAVYRSALLLGGERRFVLANSGHIQSIINPPGNPKAYYLANPK
LSSDPRAWFHDAKRSEGSWWPLWLEWITARSGLLKAPRTELGNATYPPLG
PAPGTYVLTR

Nucleotide sequence of phaC gene (SEQ ID NO: 8)

atgacagacaaaccggccaaaggatcgacaacgctccccgccacccgcat
gaacgtgcagaacgccatcctcggcctgcgcggccgcgacctgctttcca
cgctgcgcaacgtcggccgccacggcctgcgccacccgctgcataccgcg
catcatctgctggcgcttggcgggcagttggggcgggtgatgctggggga
cacgccctaccagccgaacccgcgtgacgcacgcttcagtgacccgacct
ggagccagaacccgttctaccgccgcggcctgcaagcctatctggcctgg
cagaagcagacacgccagtggatcgatgaaagccatttgaacgacgatga
tcgagcccgcgcccacttcctgttcaacctgatcaacgatgcgctggcgc
ccagcaactcactgctcaatccgttggcggtcaaggagctgttcaacacc
ggcggccagagcctggtgcgcggcgtggctcacctgctcgacgacctgcg
tcacaacgatgggctgcctcgtcaggtggacgagcgcgccttcgaagtgg
gcgttaacctggccgcaacccctggcgcagtggtatttcgcaacgagctg
ctggagctgatccagtactcgccgatgagcgaaaagcagcacgcacgccc
actgctggtcgtgccgcctcagatcaacaagttctacatcttcgacctca
gcgcgaccaacagcttcgtccagtacatgctcaaaagcggcttgcaggtg
ttcatggtcagctggcgcaaccccgacccacgccaccgtgaatggggcct
ttccagctatgtgcaagccctggaggaagcgctcaatgcctgccgcagta
tcagcggcaaccgcgaccccaacctgatgggtgcctgtgccggcggcctg
accatggccgcactgcaaggccatctgcaagccaagaagcaattgcgccg
ggtgcgcagtgccacgtatctggtcagcttgctggacagcaagttcgaaa
gcccggccagcctgttcgccgatgagcagaccatcgaagcggccaagcga
cgctcctatcagcgtggcgtgctggacggtggtgaagtggcgcggatctt
cgcctggatgcggcccaacgacctgatctggaactactgggtaaacaact
acctgctcggcaagacaccgccggcgttcgacatcctgtactggaatgcc
gacagcacgcgcctgcccgccgcgctgcatggcgacctgctggagttttt
caagctcaaccccttgacctacgcgtccgggctggaggtgtgcggtacgc
cgatcgacctgcagcaggtcaatatcgacagctttaccgtggccggcagc
aacgaccacatcacaccatgggatgcggtgtaccgctcggccttgctgct
gggtggcgagcggcgcttcgtgctggccaacagcgggcatatccagagca
tcatcaacccgccaggcaaccccaaggcctactacctggccaaccccaag
ctgagcagcgacccacgcgcctggttccacgacgccaagcgcagtgaagg
cagctggtggccgttgtggctggagtggatcaccgcacgctccggcctgc
tcaaggcaccgcgtactgaactgggcaacgccacttacccaccgctaggc
cccgcgccaggcacctacgtgctgacccgatga

Amino acid sequence of PhaJ enzyme (SEQ ID NO: 9)

MSQVTNTPYEALEVGQKAEYKKSVEERDIQLFAAMSGDHNPVHLDAEFAA
KSMFRERIAHGMFSGALISAAVACTLPGPGTIYLGQQMSFQKPVKIGDTL
TVRLEILEKLPKFKVRIATNVYNQNDELVVAGEAEILAPRKQQTVELVSP
PNFVAS

Nucleotide sequence of phaJ gene (SEQ ID NO: 10)

atgtcccaggtcaccaacacgccttacgaagccctcgaagtcggccagaa
ggccgagtacaagaagtccgttgaagaacgcgacatccagctgttcgccg
cgatgtccggtgaccacaacccggttcacctggatgccgagttcgccgcc
aagagcatgttccgcgaacgtattgcccatggcatgttcagcggcgcgct
gatcagtgcggcagtggcctgcaccctgcctggccctggcaccatctacc
tgggccagcagatgagcttccagaaaccggtgaaaatcggcgacaccctg
accgtgcgcctggagatcctcgagaagctgcccaagttcaaggtgcgcat
cgccaccaatgtgtacaaccagaacgacgagctggtggtcgcgggtgaag
ccgagatcctggcgccgcgcaagcagcagaccgtcgagctggtatcgccg
ccgaactttgtggcgagctga

As a result, as shown in FIG. 12, it was confirmed that medium-chain-length polyhydroxyalkanoates were successfully produced by fed-batch culture of the recombinant strain. This polymer was analyzed to have an average molecular weight (Mw) of 857 kD and a polydispersity of 2.18. This molecular weight is the highest among MCL-PHAS produced from glucose.

A previous study reported that MCL-PHAs with the molecular weight of 630 kDa was achieved in recombinant Pseudomonas aeruginosa using glucose and oleic acid as feedstock and MCL-PHAs with 559 kDa were achieved in lard pork fat (Solaiman et al., Curr Microbiol 44:189-195, 2002). Thus, comparison with the previous study can be made. In the previous study, a strain was modified using the Pseudomonas lipA and limA genes, encoding lipase precursor and modulator proteins, respectively, so as to produce MCL-PHAs from TAGS (Solaiman et al., Curr Microbiol 44:189-195, 2002). In a similar manner, the recombinant Rhodococcus opacus strain of this example produced high-molecular-weight MCL-PHAs by hydrolyzing TAGS and overproducing fatty acids as precursors for monomers (FIGS. 20 to 24).

### Example 9: Production of Novel Vinyl-Copolymerized Polymer Through Radical System of Recombinant R. opacus PD630 Based on Artificial Lipid Peroxidation

Regarding the production of vinyl-copolymerized polymers (vinyl polymers) by microorganisms, a previous study confirmed the production of vinyl polymers using a microbial culture medium supplemented with polymer monomers, and in the above study, the reducing power of bacteria was used for metal-catalyzed radical polymerization to synthesize vinyl polymers (Fan et al., Proc. Natl. Acad. Sci. U.S.A 115, 4559-4564, 2018). A metal-free pathway was also proposed that uses bacterial electron flow to directly generate reactive oxygen species for initiating “living” radical polymerization by bacteria (Nothling et al., J Am. Chem. Soc. 143, 1, 286-293, 2021). In this study, it was proven through the introduction of the BacRAFT system that the capacity of free energy generated through the reducing power generated by microbial culture enabled in-situ vinyl polymerization (Nothling et al., J Am. Chem. Soc. 143, 1, 286˜293, 2021).

Based on a principle similar to the above-mentioned known technology, in this example, it was determined that vinyl-copolymerized polymers could be synthesized through radical polymerization of carbon-carbon double-bonded alkenes or double-bond including unsaturated fatty acids containing monomers produced by artificial lipid peroxidation, and the production of vinyl-copolymerized polymers by the recombinant strains FG FFA JC PCO, FFAdA PCO AFLO OLET, FG FFA PCO, FG FFAdA PCO AFLO OLET, and FG FFAdA PCO AFLO JC OLET was all confirmed. This is the first report of one-step fermentative vinyl-polymer biosynthesis from glucose through the recombinant metabolic pathways without additional supplementation of monomers, obviously different from the above-mentioned known technology and polymerizing the monomers.

The vinyl-copolymerized polymer in the present invention refers to a polymer produced by polymerizing monomers using free radicals and using an in vivo artificial lipid peroxidation system as a radical initiator, without being limited thereto.

The vinyl-copolymerized polymer in the present invention is a new vinyl-copolymerized polyolefins and polyesters including polyhydroxyalkanoates obtained by polymerization between a vinyl-based monomer having a carbon-carbon double bond and the polyesters including polyhydroxyalkanoates.

### Culture Conditions

Fed-batch culture was performed at 30° C. in a 5-L MARADO-05D-PS fermentor (BioCNS) containing 1.8-L MC medium. A culture (0.3 L) was prepared by placing 1 mL of a culture (obtained by culturing in 5 mL of LB medium and incubating the culture 24 to 48 hours after inoculation) in a 250-ml Erlenmeyer flask containing 100 mL of LB medium and culturing the same for about 24 to 48 hours. The initial OD at 600 nm after inoculation was about 0.5 to 1. The initial pH of the medium was set at 6.4, then adjusted to 7.0 after 24 hours, and kept constant at 7.0 by adding 5M NaOH. Air was continuously added through a 0.2-μm HEPA filter (Millipore) at a rate of 2 L/min. The dissolved oxygen concentration was maintained at 40% air saturation by automatically adjusting the air and pure oxygen flow rates to a constant total gas flow rate of 1 vvm at an initial agitation speed of 300 rpm. The agitation speed was automatically adjusted up to 700 rpm to maintain the dissolved oxygen concentration at 40% air saturation. For fed-batch culture, when the residual glucose concentration in the bioreactor was about 15 g/L, 100-mL feed solution including 80 g of glucose and 1 g of MgSO₄·7H₂O was manually added. After acetamide was fed, a strategy of adding glucose in proportion to the glucose consumption rate of each recombinant strain was used. Acetamide was used as an inducer to overexpress intracellular lipase and was added to a final concentration of 0.17 M. Antibiotics were selectively supplemented at the following concentrations when necessary: 50 mg/L kanamycin; 100 mg/L streptomycin. Antifoam 204 (Sigma-Aldrich) was manually added to each vessel to suppress foam formation.

### Culture Medium Component

Medium component per liter: 40 g glucose, 3.3088 g KH₂PO₄, 7.9552 g K₂HPO₄, 14.2 g (NH₄)₂SO₄, 2 g MgSO₄7H₂O, 2.86 mg H₃BO₄, 15 mg CaCl₂, 1 ml stock A solution, and 1 ml of trace metal solution. Stock A solution components per liter: 2 g NaMoO₄2H₂0 and 5 g FeNaEDTA. Trace metal solution components per liter: 0.5 g FeSO₄7H₂O, 0.4 g ZnSO₄H₂O, 0.02 g MnSO₄H₂O, 0.01 g NiCl₂6H₂0, 0.05 g CuSO₄5H₂O, 0.01 g MnCl₂, and 0.05 g CoCl₂6H₂O.

As a result, as shown in FIGS. 15 and 16, the presence of n-alkene oligomers of 5.31 kDa and 1.36 kDa Mw in the culture resulting from fed-batch culture was confirmed by GPC analysis, and the biosynthesis of middle-chain-length polyhydroxyalkanoates was also confirmed to be a polymer having an ultra-high molecular weight of 857 KDa, obtained through polymerization of the corresponding unsaturated double bond containing monomers such as alkenes.

This result is the first report of achieving bacterial synthesis of a vinyl polymer without supplementing related unsaturated monomers. This polymer has a thermal degradation temperature similar to that of polyethylene/polypropylene, kinds of conventional polyolefins, and thus may be used as an environmentally-friendly melt adhesive or substitute to polyesters or polyolefins having relatively high thermal degradation stability compared to existing medium-chain-length polyhydroxyalkanoates.

### INDUSTRIAL APPLICABILITY

Using the lipid peroxidation system of the present invention, it is possible to produce ultra-high concentrations of free fatty acids through artificial lipid peroxidation without cell death, and it is also possible to produce biofuels and biopolymers from these free fatty acids. In addition, the artificial lipid peroxidation system according to the present invention can further promote carbon dioxide fixation by increasing intracellular redox energy density, making improved production of biofuels and biopolymers possible.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### SEQUENCE LISTING FREE TEXT

Electronic file attached

1. A recombinant bacterium with induced cell membrane lipid peroxidation ability, wherein a heterologous peroxidase-encoding gene is introduced thereinto.

2. The recombinant bacterium of claim 1, wherein the peroxidase has an EC number of EC 1.11.1.14.

3. The recombinant bacterium of claim 1, wherein the recombinant bacterium is a Gram-positive bacterium.

4. The recombinant bacterium of claim 1, wherein the bacterium is selected from the group consisting of Gram-positive bacteria having mycolic acid as a cell membrane component, including Rhodococcus, Corynebacterium, Mycobacterium, and Gordonia.

5. The recombinant bacterium of claim 1, wherein an acyl-CoA synthetase-encoding gene is deleted.

6. The recombinant bacterium of claim 1, wherein a promoter of a monoacylglycerol (MAG) lipase-encoding gene is replaced with an inducible promoter.

7. The recombinant bacterium of claim 1, wherein a 4-dichlorophenol 6-monooxygenase-encoding gene or a 2-dehydropantoate 2-reductase-encoding gene is further deleted.

8. The recombinant bacterium of claim 1, wherein a laccase-like multicopper oxidase (LMCO)-encoding gene is further introduced.

9. The recombinant bacterium of claim 1, wherein glucose adaptive evolution has been further performed thereon.

10. The recombinant bacterium of claim 1, wherein a gene encoding an enzyme that converts fatty acids into 1-alkenes is further introduced.

11. The recombinant bacterium of claim 1, wherein a gene encoding an enzyme that degrades hydrocarbons into 1-alcohols is deleted.

12. The recombinant bacterium of claim 1, wherein a gene encoding a medium-chain-length (MCL) polyhydroxyalkanoate synthase and a gene encoding (R)-specific enoyl-CoA hydratase are further introduced.

13. The recombinant bacterium of claim 1, which has a reduced cell membrane thickness compared to a parent strain.

14. A method of producing free fatty acids by cell membrane lipid peroxidation comprising the steps of:
(a) culturing the recombinant bacterium of claim 1 to produce free fatty acids by cell membrane lipid peroxidation; and
(b) recovering the produced free fatty acids.

15. A method of producing triacylglycerols by cell membrane lipid peroxidation comprising the steps of:
(a) culturing the recombinant bacterium of claim 1 to overproduce triacylglycerols by cell membrane lipid peroxidation; and
(b) recovering the produced triacylglycerols.

16. A method for producing hydrocarbons comprising the steps of:
(a) culturing the recombinant bacterium of claim 1 to produce hydrocarbons; and
(b) recovering the produced hydrocarbons.

17. The method of claim 15, wherein step (a) comprises additionally supplementing carbon dioxide.

18. A method for producing polyolefins comprising the steps of:
(a) culturing the recombinant bacterium of claim 1 to produce polyolefins; and
(b) recovering the produced polyolefins.

19. The method of claim 16, wherein step (a) comprises additionally supplementing carbon dioxide.

20. A method for producing polyhydroxyalkanoates comprising the steps of:
(a) culturing the recombinant bacterium of claim 1 to produce polyhydroxyalkanoates; and
(b) recovering the produced polyhydroxyalkanoates.

21. A method for producing vinyl polymers including vinyl-copolymerized polyhydroxyalkanoates comprising the steps of:
(a) culturing the recombinant bacterium of claim 1 to produce vinyl polymers including vinyl-copolymerized polyhydroxyalkanoates; and
(b) recovering the produced vinyl polymers including vinyl-copolymerized polyhydroxyalkanoates.

22.-43. (canceled)

44. The recombinant bacterium of claim 1, wherein the peroxidase is derived from Phanerochaete carnosa.

45. The recombinant bacterium of claim 44, wherein the peroxidase has the amino acid sequence of SEQ ID NO: 1.
